(19) United States
(12) Patent Application Publication
(10) Pub. No.: US 2012/0184001 A1
(43) Pub. Date: Jul. 19, 2012

(54) SYSTEMS AND METHODS FOR SUSTAINABLE AQUACULTURE

(75) Inventors: David Stephen, Davis, CA (US); Benjamin Chiau-pin Wu, San Ramon, CA (US)

(73) Assignee: LiveFuels, Inc., Menlo Park, CA (US)

(21) Appl. No.: 13/319,999

(22) PCT Filed: May 13, 2010
(86) PCT No.: PCT/US10/34640

§ 371 Date: Dec. 16, 2011

Related U.S. Application Data
(60) Provisional application No. 61/178,888, filed on May 15, 2009.

(51) Int. Cl. C12P 7/64; C07K 1/14; C10L 1/188; A01K 61/00; A23D 9/00; A01K 63/04; A01K 61/02; A23K 1/10; A23D 7/00; C11B 1/00; A01K 63/00

(52) U.S. Cl. 435/134; 554/8; 530/412; 44/385; 119/200; 119/207; 119/223; 119/226; 119/230; 426/665; 426/417; 426/33

(57) ABSTRACT

Provided herein are systems and methods for sustainable aquaculture. The methods provided herein allows recovery and/or recycling of autochthonous nutrients in fish farming, and recovery of allochthonous nutrients present in eutrophic water. The systems provided herein comprises two closely spaced cages, an array of rotary panels that regulates the flow of matters between the two cages; and a means for producing a directional water current.

This application claims the benefit of U.S. Provisional Application No. 61/178,888, filed May 15, 2009, which is incorporated by reference in its entirety.

### 1. INTRODUCTION

Provided herein are systems and methods for sustainable aquaculture.

### 2. BACKGROUND OF THE INVENTION

By 2040, the number of humans on earth is expected to reach 9 billion, more than three times greater than the population of 1960. In 2008, there were approximately 6.6 billion humans; 305 million human live in the United States (U.S. Census Bureau, Population Division, International Database, December 2008). The increasing human population will eventually deplete remaining finite or non-renewable resources; however, our future survival will depend on sustaining the living (renewable) resources at optimal levels, particularly natural food systems and agricultural production.

In the last three decades, unsustainable fishing practices have left a shrinking resource base which now threatens global food security. The Food and Agriculture Organization of the United Nations estimates that 11 of the world's 15 major fishing areas and 69 percent of the world's major fish species are in decline and in need of urgent conservation. The challenge now is to keep fish production on the rise to meet the increasing protein needs of a growing global population, while at the same time allowing overfished populations to recover and preventing other species from joining the list of the overfished stocks.

Aquaculture has been the fastest growing food production sector for the last decade, and there is significant potential for continued expansion and growth, particularly for offshore marine aquaculture. However, aquaculture is faced with the challenges of sustainable development in a world where environmental impacts of urban development, industries, intensive agriculture and animal husbandry have already seriously impinged on the resilience of the planet's life support systems. In general, aquaculture is practiced where land and water are available. This results in a concentration of fish farms around lakes, deltas, and coastal margins, which often amplifies the problem of environmental impacts from discharged effluents. Fish farms also tend to be located close to economically vital regions supporting large urban and industrial centers where competing and conflicting uses of natural resources are invariably present. There is also an increase in recreational use of these bodies of water which demands higher water quality. Nitrogen loads generated from the sewage effluent of growing urban agglomerations and agricultural runoffs severely limits the discharge of aquaculture effluent into these aquatic environments.

Nutrient over-enrichment leads to algal bloom resulting in changes in biodiversity and loss of habitats (e.g., decline of coral reef), and appearance of “dead zones” where most animals die of hypoxia. Changes in the environmental quality of lakes and coastal waters adversely impacts population structures and biodiversity. The Gulf of Mexico has a seasonal hypoxic zone that forms every year in late summer. Its size varies, in 2000, it was less than 5,000 km², while in 2002, it was approximately 22,000 km² (or the size of the state of Massachusetts). Of the 415 areas around the world identified as experiencing some form of eutrophication, 169 are hypoxic (Selman et al., Eutrophication and hypoxia in coastal areas: a global assessment of the state of knowledge. World Resource Institute Policy Note, March 2008.) In nutrient loaded waters, blooms of toxic algae are known to cause mass mortalities of wild fish and shellfish, birds and even mammals. Fish farms that use these waters are also impacted. Humans develop paralysis, diarrhea, and amnesia when contaminated seafood is consumed. In 1997, hundreds of dead fish were found in Chesapeake Bay and Pokomoke River near Maryland in the United States, resulting in closures of waterways and ban on seafood from the area. The economic loss suffered by the local seafood industry was estimated to be US$43 million (Lipton, D. W. 1998. Pfiesteria's economic impact on seafood industry sales and recreational fishing. Pfiesteria: where do we go from here? Economics of Policy Options for Nutrient Management and Dinoflagellates Conference. University of Maryland, Department of Agriculture and Natural Resources, College Park). In 1998, one red tide event wiped out 90 percent of Hong Kong's fish farms and resulted in an estimated economic loss of US$40 million (Lu, S., and I. J. Hodgkiss. 2004. “Harmful algal bloom causative collected from Hong Kong waters.” Hydrobiologia, 512(1-3): 231-238). The aquaculture industry will surely suffer if the quality of water it uses is not managed and protected and the quality of water it discharges must therefore comply with standards set for all users so that water quality and biodiversity is sustained.

While nutrient input by the aquaculture industry is small compared to inputs from terrestrial agriculture, total wastes from many intensive fish farms can become substantial and thus have adverse impacts in closed water bodies and poorly flushed coastal embayments. To reduce the environmental impacts of aquaculture, technology-based efforts are needed to improve resource utilization and to develop appropriate waste management strategies. In this respect, provided herein are systems and methods for sustainable cage-based aquaculture in water bodies or offshore open waters or water surface or subsurface based production facilities for the farming of fish, shellfish, seaweeds and other aquatic organisms.

### 3. SUMMARY OF THE INVENTION

Provided herein are systems and methods for sustainable cage-based aquaculture. The aquaculture system comprises two compartments separated by a means for regulating flow of matters between the two compartments. In one embodiment, the system comprises two closely-disposed meshed cages, an array of spaced rotary panels that regulate the flow of matters between the two cages; and a means for driving water from one cage to another. One of the two cages, referred to herein as an inner cage, is disposed inside an outer cage. In addition to the interior of the inner cage, the exterior surface of the inner cage and the interior surface of the outer cage define another space for culturing secondary species of fish and/or shellfish and or seaweeds or other aquatic organisms. The two cages are independent, prismatic, cylindrical or funnel-shaped, and can be aligned coaxially. The cages can be deployed individually or in a cluster in a body of water.

In another embodiment, in addition to the inner and outer cages, a third cage or a receptacle is closely disposed to the bottom of the inner cage and/or the bottom of the outer cage, such that solid wastes in the inner cage and/or outer cage can be directed to and collected in the third cage or receptacle. The receptacle is connected to ducts and a pump for conveying the solid wastes to a surface-based receiver.

The array of panels are disposed vertically or horizontally in the cage and are rotatable about itself on an axis. This central feature of the inner cage can also be used in an outer cage. When the panels are rotated to an angular position that is about parallel to the side wall, a barrier to flow in and out of the cage is formed, i.e., the panels are in a closed position. When the panels are rotated to an angular position that is about perpendicular to the side wall, the flow is minimally obstructed by the panels, i.e., the panels are in an open position. Any intermediate angular position would allow a certain amount of water to flow pass the panels.

In certain embodiments, the panels are made of materials that are impermeable to water, or impermeable to solid waste produced in the cage. The bottom of the inner cage comprises at least one meshed aperture through which solid wastes in the inner cage pass into the outer cage but prevent the escape of fish to the outer cage. The outer cage may also comprise an array of panels, similar to those of the inner cage. The means for driving water from one cage to another can be a device that produces a directional water current, such as but not limited to, a water pump, an air pump, a venturi aerator, an agitator, a propeller, a pressurized water and/or air manifold.

In a specific embodiment, the aquaculture system comprises a buoyant, cylindrical or funnel-shaped, inner cage which contains piscivorous fishes or fishes fed with aquaculture feed, said inner cage being disposed coaxially inside a buoyant outer cage which contains algae, and planktivorous fishes and/or planktivorous shellfishes in a space defined by exterior surfaces of the inner cage and interior surfaces of the outer cage. The inner cage comprise an annular array of rotary panels disposed within the confines of the inner cage next to the meshed side walls. The panels are rotated about a vertical axis coordinately to regulate flow of water/fluid/matters in and out of the inner cage. This system also include one or more columnar air and water jet manifolds which generate an unidirectional centripetal current that forces heavier suspended solids to move towards the bottom centre of the inner cage and to fall into the third cage or receptacle. Water with dissolved and fine particulate wastes from the inner cage moves to the outer cage through the gaps between the panels. The same device(s) can also be used to draw aerated water into the inner cage.

Provided herein are methods of aquaculture which comprise providing a system comprising a buoyant inner cage disposed inside a buoyant outer cage, and an array of spaced rotary panels disposed between the two cages and that regulate the flow of fluids/matters in and out of the inner cage. The inner cage contains piscivorous fishes and fishes that are fed conventional formulated/pelletized aquaculture feed (feed-fed fishes), while the outer cage contains algae, and planktivorous organisms in a space defined by exterior surfaces of the inner cage and interior surfaces of the outer cage. The rotary panels are rotated to the closed position during periods when the fishes in the inner cage are feeding or defecating so as to reduce or prevent passage of solid wastes from the inner cage to the outer cage through gaps between the panels, or from the outer cage to ambient water (if the outer cage also comprises rotary panels). Solid wastes that settle to the bottom, such as dead fish, uneaten food and feces, fall to the bottom and exit the inner cage through the aperture and are collected in a receptacle. When solid wastes are removed from the inner cage, the panels are rotated to an open position. Dissolved waste and finer particulate waste are flushed from the inner cage through the gaps between the rotary panels to the outer cage where the micro or macro algae consumes dissolved nutrients and filter feeding fish (planktivorous fish) and shellfish “filter out” or feed on finer suspended particulates and also on the microscopic algae or plankton.

To reduce the use of fishmeal made from captured wild fish stocks, the method can further comprise feeding the piscivorous fishes or feed-fed fishes in the inner cage with live planktivorous fishes from the outer cage, or with fish feed that comprises processed planktivorous fishes from the outer cage. Where the system is disposed in a body of eutrophic water, the algae grows in the outer cage using nutrients in the body of eutrophic water, thereby recovering allochthonous nutrients from the surrounding eutrophic water. In one embodiment, the planktivorous fishes and/or planktivorous shellfishes in the outer cage are fed with an algal composition, in addition to the algae in the water within the confines of the cage. The method further comprises harvesting the piscivorous fishes and/or feed-fed fishes, or the piscivorous fishes as well as the planktivorous fishes and/or the planktivorous shellfish. The harvested fishes and/or shellfishes can be sold as human food, used as fish feed directly or after processing, or use as an energy feedstock or an industrial feedstock. In certain embodiments, the aquaculture system comprises detritivorous fishes that consume solid wastes collected in the bottom of the inner cage and/or the outer cage, thereby reducing wasting of feed and collection of solid waste in the receptacle. The detritivorous fishes can be harvested for sale, for processing into fish feed, energy feedstock or industrial feedstock.

Certain embodiments provided herein can be used in commercial fish farming or in bioremediation. The cages can be used to reduce the amount of wastes that are released into ambient water by aquaculture operations. The cages can be deployed in the path of nutrient run-off, in eutrophic zones or in areas with upwelling, to recover nutrients in the water.

### 4. DESCRIPTION OF THE FIGURES

FIG. 1: An inner cage 101 and an outer cage 102 in a body of water 100. The inner cage comprises meshed side walls 110 and panels (not shown). Piscisvores and/or feed-fed fishes 201 are cultured in the inner cage. Planktivores 202 and optionally herbivores and detritivores, are cultured in the outer cage. Autochthonous solid wastes produced in the inner cage 301 is directed through the aperture 103 and collected in the receptacle 104. Autochthonous dissolved waste 304 flows outward from the inner cage. Autochthonous solid wastes produced in the outer cage 302 is directed through the aperture 105 and collected in the receptacle 106. Allochthonous nutrients, plankton, and algae 303 enter the outer cage through meshed side walls (not shown).

FIG. 2: Plan view shows inner cage 101 enclosed within outer cage 102; frame for meshed side wall 111, rotary panels 112, frame for rotary panels 113, columnar air and water diffuser 116, directional water flow, and central aperture 103. Cut-away view of inner cage 101: meshed side wall 110, frame for side wall 111, rotary panels 112 in open position, frame for rotary panels 113, columnar air and water diffuser 116. Autochthonous dissolved waste 304 flows outward from the inner cage through gaps 115 between rotary panels in open position.

FIG. 3A. Panels in open position: angular position 117 of rotary panels 112 approaches or is perpendicular to tangent 118; the open position allows water flow 304 from inner cage 101 to outer cage 102, through gap 115 between rotary panels 112, hinged at 114 and supported by frame 113, inside meshed side wall supported by its frame 111. FIG. 3B. Panels in closed position: angular position 117 of rotary panels 112 is small and acute when measured against tangent 118, the panels 112 are almost parallel to the side wall.

### 5. DETAILED DESCRIPTION OF THE INVENTION

Provided herein are systems and methods for fish farming in a sustainable manner, and for bioremediation of eutrophic zones. Certain embodiments provided herein are based on nutrient recycling and recovery, and multitrophic polyculture. Certain embodiments provided herein are developed to alleviate the problems with autochthonous and allochthonous nutrients in aquaculture. It can also be used in offshore aquaculture where deep sea nutrients are drawn up by natural or artificial upwelling to boost primary production.

The aquaculture approach provided herein mimics parts of a natural trophic system or food chain, wherein primary producers, such as algae, generate biomass using sunlight and nutrients in the water. Planktivores and herbivores, consume the primary producers; higher trophic level consumers, such as piscivores, in turn consume those lower in the food chain. Detritivores derive energy from solid waste, including uneaten fish feed, excrement, and dead organisms. Accordingly, the methods of aquaculture provided herein involve culturing fishes and shellfishes that occupy different trophic levels in the same aquaculture system.

This aquaculture system is designed such that fishes and/or shellfishes of different trophic levels are contained in separate compartments but in close proximity. In particular, the system comprises at least two compartments situated in a body of water, a first cage containing fishes that consume other fishes, i.e., piscivores, or fishes that are fed aquaculture feeds; and a second compartment containing fishes and/or shellfishes that consume primary producers, such as plankton and algae, i.e., planktivores and herbivores. An array of rotary panels, disposed in between the first and second compartments, regulates the flow of matter between the two compartments. In addition, the aquaculture system comprises a source of algae; and optionally, fishes and/or shellfishes that consume detritus and solid waste. Certain embodiments provided herein are described below, without limitations, in terms of discrete cages, however, systems and methods can also be implemented by any means for confining fishes and/or shellfishes in a compartment within a body of water, for example, by using partitions and nets.

The fishes that are cultured in the first cage (also referred to herein as inner cage) include piscivores and/or fishes that are routinely fed with formulated or pelletized aquaculture feeds in conventional fish farms. They are generally of a higher economic value than the fish in the second cage. The piscivores and fishes that are fed with aquaculture feeds in conventional fish farms, herein referred to as “feed-fed fishes,” are harvested and marketed as human food. The planktivores and herbivores in the second cage (also referred to herein as outer cage), depending on the species, can also be marketed as human food. The planktivores and herbivores can be used as live feed for the piscivores or fishes in the first cage/inner cage. Alternatively, the planktivores and herbivores can be processed into fishmeal which is fed to the piscivores or feed-fed fishes in the first cage/inner cage. In certain embodiments, fishes and/or shellfishes that can consume detritus and solid waste, i.e., detritivores, can be cultured in the second cage/outer cage with the planktivores and herbivores. Alternatively, the detritivores can be cultured in a separate third cage. Detritivores can also be harvested and marketed as human food, or used as live feed or after processing into fishmeal as feed for the piscivores or fishes in the first cage/inner cage. The use of planktivores, herbivores, and/or detritivores of the system as fish feed for the piscivores or feed-fed fishes in the first cage/inner cage can reduce the dependence on unsustainable fish meal produced from wild fish stocks and unsuitable feed stocks from rendered terrestrial animals. Fishes that are not sold as human food or used as fish meal can also be used for producing biofuels and oleochemical feedstocks. The populations of planktivores, herbivores, detritivores, and piscivores in the cages provided herein generally comprises a single species in each trophic category or multiples species in each trophic category for example, two or more piscivorous species may be cultured in the first or inner cage; two or more planktivorous species may be cultured in the second or outer cage; two or more detritivorous species; two or more species of shellfishes, or two or more species of seaweeds may be cultured in these cages. The fishes provided herein are described in details in Section 5.2.

The two cages of the system are configured such that wastes produced by the fishes in the first cage/inner cage are used by algae growing in the water within the confines of these cages. The resulting algal biomass can be consumed by fishes and/or shellfishes in the second cage/outer cage. In embodiments where three cages are used, the third cage containing detritivores is configured so that detritus, such as uneaten food and solid wastes, are directed to the third cage and consumed by the detritivores. Certain embodiments provide the recycling of autochthonous nutrients within this aquaculture system. It is preferred that the water currents are not so strong that nutrients are being drawn out of the outer cage at a rate faster than nutrients are being captured by the growing microalgae and macroalgae. In embodiments where detritivores are provided in the system, they can be cultured along with the planktivores and herbivores, and even the piscivores. When detritivores are placed in the same enclosure or cage, with piscivorous species, the detritivores are so selected to ensure that they are not favored as food by the piscivores. Detritivores are bottom feeders with mouth located venterally and they do not compete with piscivores, planktivores and herbivores. Detritivores tend to stay close to the floor of the cages where solid waste settles.

In a preferred embodiment, the first cage containing piscivores or fishes that are fed formulated/pelletized aquaculture feed is disposed inside the second cage containing planktivores and herbivores. Placement of planktivores and herbivores inside the second cage is advantageous since the autochthonous nutrients can be recycled more efficiently. Detailed description of the design of such nesting cages are provided in Section 5.1.

Generally, the methods provided herein comprise (i) providing an aquaculture system comprising a first cage and a second cage that are independently buoyant and closely disposed to each other, said first cage comprising an array of spaced rotary panels, wherein said panels regulate the flow of water from the first cage to the second cage by increasing or decreasing gaps between neighboring rotary panels; (ii) providing piscivorous fishes in the inner cage, and algae and planktivorous fishes and/or shellfishes in a space defined by exterior surfaces of the inner cage and interior surfaces of the outer cage; (iii) rotating the panels periodically, each panel rotatable about a vertical or horizontal axis, to an angular position that reduces or prevents passage of solid wastes from the inner cage to the outer cage through gaps between the panels; and (iv) rotating the panels periodically to an angular position that increases or allows flow of water from the inner cage to the outer cage through the gaps between the panels.

Algae are provided in the system as feed for the planktivores and herbivores in several ways. In one embodiment, algae is cultured in enclosures separate from the cages where the fishes of interest are cultured. The algae enclosure may itself contain other fishes. Algae from this separate enclosure are introduced into the cages, the second cage, or the body of water within the confines of the cages. In another embodiment, the algae occur naturally in the body of water where the cages are situated. Under oligotrophic conditions, appropriate fertilizers are added to the water to stimulate and sustain the growth of algae. Nutrients can also be derived from natural or artificial upwelling that brings nutrients near the bottom of the water column up to the photic zone. In embodiments where the aquaculture system is situated in eutrophic water, there is usually an abundance of algae, and fertilization of the water may be necessary only to augment certain limiting nutrients. The terms “eutrophic” and “eutrophication” are used herein to describe the presence of an abundance of nutrients in a body of water, i.e., nutrient enrichment, with productivity greater than about 300 g C m⁻² year⁻¹. Under eutrophic conditions, the aquaculture system can recover allochthonous nutrients through the production of algal biomass and fish biomass. Depending on the circumstances, the aquaculture system can also reduce the risk of harmful algal bloom and/or hypoxia, and reverse some of the negative ecological impacts of eutrophication. Nutrient enrichment of the water in the inner cage involves typically metabolic wastes, feces, feed dust and crumbles, excess food, uneaten food, and dead organisms. It is contemplated that an algal composition can be prepared by mixing different algae from a plurality of algal cultures. The diversity and relative quantities of algae present in a body of water in which the cages are situated can be used to further define an aquaculture system provided herein. Algae that are useful in the embodiments are described in Section 5.3.

Currently, the aquaculture industry is engaged mostly in intensive monoculture, wherein a single fish species of high economic value is cultured at a density higher than that which occurs in a natural body of water. The ponds are typically stocked at a high density of omnivores or carnivores which are fed with pelletized aquaculture feeds. These feeds are made from fishmeal and/or feedstock from rendered terrestrial animals. Higher stocking density of fish requires proportionally higher rates of feeding which in turn causes a higher oxygen demand and hence enclosures must be aerated or oxygenated throughout much of the day and night. It has been estimated that up to 22 kg of wild-caught fish is needed to produce just 1 kg of farmed tuna, or 4 kg of wild-caught fish is needed to produce 1 kg of farmed salmon.

The high demand for fishmeal in terrestrial animal farming and aquaculture is causing overfishing of natural stocks that is unsustainable and threatens collapse of the natural fish population. These stocks comprises planktivores that form very large shoals in the range of hundreds of tons. They also serve as forage/food species for higher trophic level predators such as solitary species like sharks, marlins and whales, and those that form important fisheries, for example, tunas. The methods provided herein that planktivores produced by the system are used as live food or are rendered into fish meal and fish oil for incorporation in formulated or palletized feeds, thus significantly reducing dependence on fish oil and fish meal made from unsustainable fishing of wild stocks.

Intensive monoculture typically deploys clusters of cages in a body of water. One of the main problems with aquaculture is the diffusion of waste nutrients into the surrounding water. The concentration of the cages amplifies the undesirable effect of the waste nutrients. Aquaculture wastes include uneaten food, fecal, and urinary products of the fish. As much as 20% of the food provided to cultured fish is not eaten. Not only is the feed protein from fishmeal unsustainable as noted above, the feed costs may be as high as 50% of the production fish cost and pollution effects can add significantly to mitigation costs. Mass balance studies based on feed quantity, feed qualities, food conversion ratio, digestibility, and fecal composition, can be used to estimate the release of nutrients to the environment.

Excretory products, such as ammonia, are dispersed in the water column by currents while solid wastes, such as uneaten food and feces, settle towards the bottom of the sea or lake. During sedimentation, some of the solid wastes are consumed by various organisms, and break down into finer particles. Depending on physical properties of the solid wastes, temperature, depth of water, turbulence, and microbial consumption, nutrients are solubilized in the process and released into the water. Nutrients are also released from sedimented solid waste. Respiration of bacteria in the water column and sediments that consume the solid wastes causes a loss of oxygen. If the loss is not offset by the introduction of additional oxygen by photosynthesis or mixing with oxygen-rich water, then hypoxia or anoxia occurs. Dissolved oxygen less than about 2.0 to 3.0 mg per liter is referred to herein as hypoxia. Anoxia is a form of hypoxia when biologically useable dissolved oxygen is completely absent and this leads to mass mortalities. Benthic hypoxia may change migration, habitat, and benthic communities structure.

Eutrophication is often readily detectable in freshwater cage sites set in lakes where currents are slow and where dilution is limited. It can also be detected by changes in dissolved oxygen (DO), chemical oxygen demand (COD), biological oxygen demand (BOD), and turbidity or transparency. In marine intertidal sites, dilution of nutrients is much more rapid, eutrophic effects are apparent only during slack tides. Immediately under the cages, a hypoxic zone can develop and emigration of macrobenthic organisms generally follows. The area of benthos affected by autochthonous nutrients from intensive cage farming typically extends 20 to 50 meters beyond the cages, although in some sites, because of poor management and hydrographic conditions, effects are evident up to 150 meters from the cages. Certain embodiments provide the uptake of autochthonous nutrients by culturing a plurality of organisms of different trophic levels in the system.

Beside the self polluting effect of poorly managed cage culture practices, other nutrients may also be present due to remote sources. Agriculture, human sewage, urban run-off, industrial effluent, and fossil fuel combustions are the most common allochthonous sources. The intensive use of commercial fertilizers over the last 50 years has increased crop yield but nutrients not absorbed by crops find their way into local rivers and lakes, and are generally carried downstream to the estuaries, and eventually to the sea. Human activities have resulted in the near doubling of nitrogen and tripling of phosphorus flows to confined water bodies and open coastal environments when compared to natural causes from weathering and land runoff. Typically, the body of affected water is plagued by algal blooms, e.g., during spring time or after a storm. Certain embodiments provide the recovery of allochthonous nutrients in eutrophic water by culturing a plurality of organisms of different trophic levels in the system.

Some of the characteristics of eutrophic water, relative to non-eutrophic water, include but are not limited to, increased phytoplankton productivity, high chlorophyll a concentration, decreased light availability to benthic zone, high epiphytic growth rate, high non-perennial macroalgae growth rate, changes in dominance from benthic algae to pelagic algae, and a shift in dominance from diatoms to dinoflagellates. Many coastal waters are shallow enough that benthic plant communities thrive where sufficient light penetrates the water column to the seafloor. Benthic vascular plants (seagrasses) and perennial macroalgae are more adapted to low nutrient environments than phytoplaktons and ephemeral macroalgae. An increase in nutrient input results in the progressive selection for fast-growing algae that are best adapted to high-nutrient conditions, at the expense of slower-growing seagrasses and perennial macroalgae. Phytoplankton biomass can reduce light penetration and epiphytic microalgae becomes more abundant on seagrass leaves in eutrophic water contributing to light attentuation. Ephemeral macroalgae, such as Ulva, Caldophora, and Chaetomorpha, can form extensive thick mats over the seagrass leading to its disappearance from the seafloor. Loss of benthic seagrasses and macroalgae will result in changes in the associated fauna. Increased sediment resuspension from tidal currents and wind fetch disturbances cause reflux of nutrients in the sediment further promoting algal blooms. The accumulation of ephemeral macroalgae is a nuisance to recreational users of beaches and waterways. Certain embodiments can be used to reverse at least some of the changes observed in the aquatic environment that results from eutrophication caused by allochthonous nutrients, e.g., the loss of seagrasses and perennial macroalgae.

Plankton species have a wide range of nutrient requirements and the plankton community composition of a coastal region can be directly changed by eutrophication. A limitation of silicates in the water restricts the growth of diatoms and/or the amount of silicon in their bodies. It has been observed that as the size of the diatom population in a water column falls, an increase in the number of flagellates is detected. Among the many species of algae that thrives in a body of water, some can produce toxins that are harmful to other organisms that either ingest these species or share the same aquatic environment. For example, the species Chaetoceros has been associated with the deaths of farmed salmon. When one or more species of algae that can produce toxins or cause harm to other marine life and humans, proliferate and become numerically dominant in an eutrophic zone, a harmful algal bloom (HAB) is formed. Certain embodiments can be used to resist the formation of a harmful algal bloom or to reduce the algal biomass and prevent the formation of blooms.

Technical and scientific terms used herein have the meanings commonly understood by one of ordinary skill in the art to which the present embodiments pertain, unless otherwise defined. Reference is made herein to various methodologies known to those of skill in the art. Publications and other materials setting forth such known methodologies to which reference is made are incorporated herein by reference in their entireties as though set forth in full. The practice of the systems and methods provided herein will employ, unless otherwise indicated, techniques of chemistry, biology, and the aquaculture industry, which are within the skill of the art. Such techniques are explained fully in the literature, e.g., Aquaculture Engineering, Odd-Ivar Lekang, 2007, Blackwell Publishing Ltd.; Handbook of Microalgal Culture, edited by Amos Richmond, 2004, Blackwell Science; Limnology: Lake and River Ecosystems, Robert G. Wetzel, 2001, Academic Press; Cage Aquaculture. M. Beveridge, third edition, 2004, Blackwell Publishing Ltd.; each of which are incorporated by reference in their entireties.

As used herein, “a” or “an” means at least one, unless clearly indicated otherwise. The term “about,” as used herein, unless otherwise indicated, refers to a value that is no more than 20% above or below the value being modified by the term. For clarity of disclosure, and not by way of limitation, the detailed description of the invention is divided into the subsections which follow.

### 5.1 Systems of the Invention

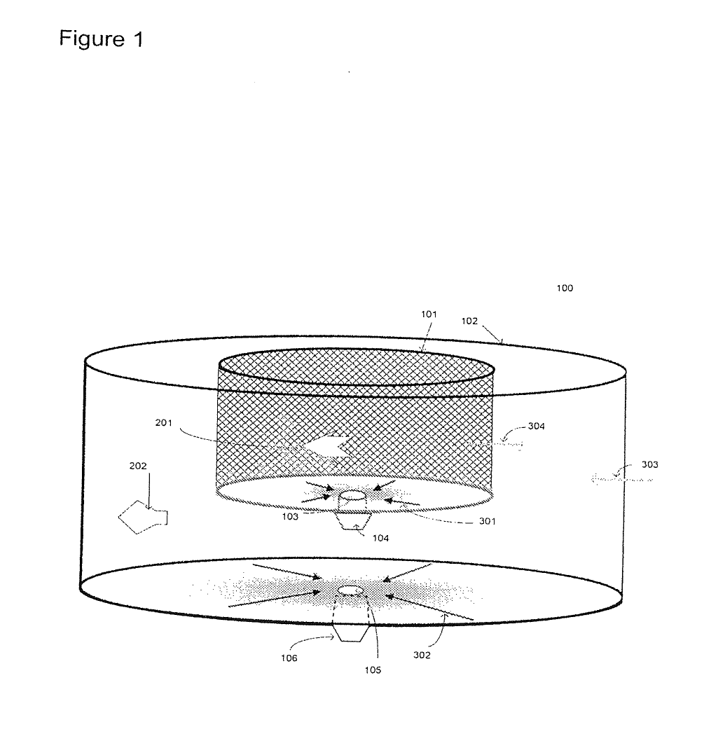

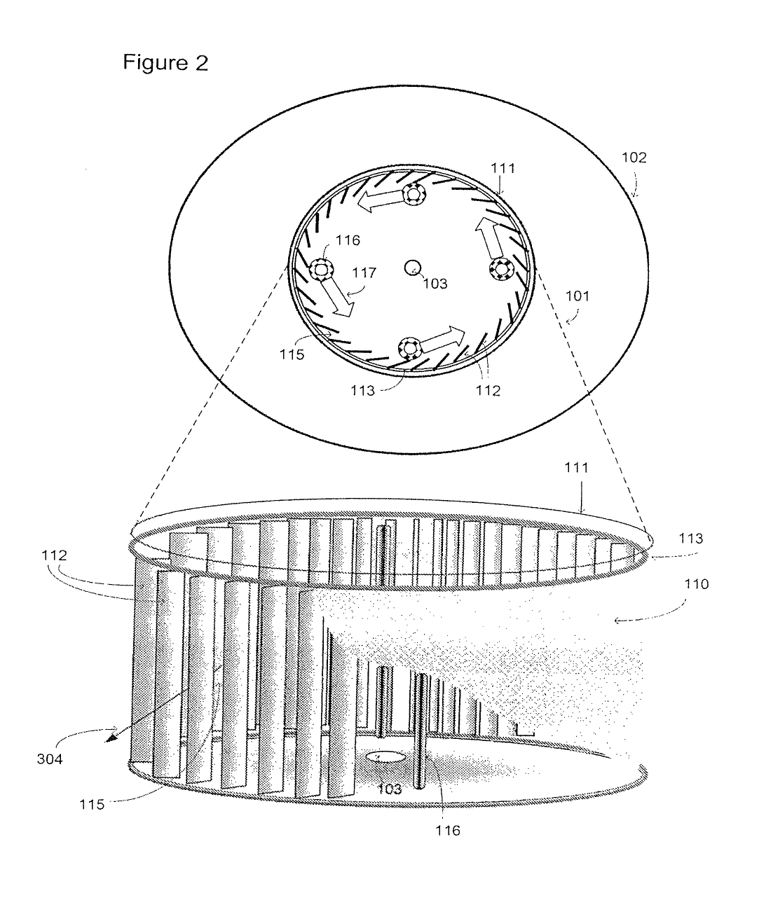

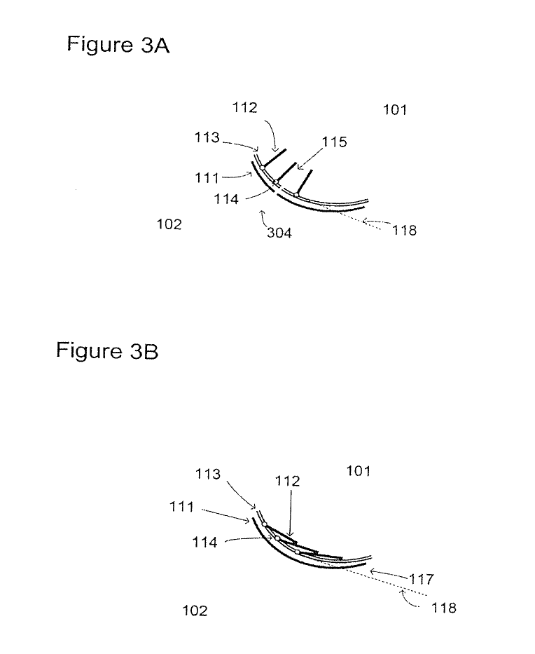

The systems provided herein comprise at least two compartments closely disposed to each other, wherein a first compartment contains piscivores and/or fishes that are fed aquaculture feeds; a second compartment contains planktivores and/or herbivores. The two compartments are so closely disposed that water can pass from one cage to another. A means for regulating fluid flow between the two compartments is also provided. Optionally, the systems comprise a third compartment which contains detritivores. Preferably, the cages are independently buoyant. In addition, the systems comprise a means for generating a directional water current that drives water mass from a compartment containing piscivores and/or feed-fed fishes to a planktivores/herbivores-containing compartment or a planktivores/herbivores-containing compartment and a detritivores-containing compartment. Without limitation, the systems provided herein are described in terms of cages, and by way of examples with reference to the accompanying drawings labeled FIGS. 1, 2, 3A and 3B.

Provided herein are a first cage that contains piscivores and/or fishes that are fed formulated/pelletized aquaculture feeds that is partially or completely disposed inside a second cage that contains planktivores and/or herbivores. An optional third cage containing detritivores can be disposed directly underneath the first and/or second cages. In a preferred embodiment, the cage containing the piscivores and/or fishes that are fed pelletized aquaculture feeds, also referred to as the inner cage, is completely enclosed by the second cage that contains planktivores and/or herbivores, also referred to as the outer cage. The inner cage can be placed anywhere inside the outer cage. Fishes and/or shellfishes in the second outer cage are contained in a space formed between interior walls of the outer cage and exterior walls of the inner cage. The meshed side walls of the cages allow water to percolate throughout the cages. The cages can independently be of any shape, preferably prismatoid (with polygonal cross-sections), and most preferably cylindrical (with a circular cross-section). The depth of the outer cage is greater than or equal to that of the inner cage. The frame of the cages provided herein can be made of any material(s) used in the construction of conventional fish cages, e.g., natural material, such as bamboo and wood; plastics, such as, high density polyethylene (HDPE); and metal, such as steel. The cages individually or as a cluster can be anchored to the seabed.

The mesh of the side walls can be flexible or rigid, and are made of natural and/or synthetic material(s), such as cotton, bamboo, wood, reeds, nylon and other plastics (e.g., polyamide, polypropylene, polyethylene, fiberglass), and metals (e.g., galvanized steel). Many of the materials are commercially available. The mesh size is selected in accordance with the dimensions of the selected organisms in the enclosures and the tensile strength of the mesh materials is selected based on size and behavior of the selected species in the enclosures and potential wild predators outside of cages that may tear the mesh. The mesh size ranges from about 1 mm, 2 mm, 4 mm, 5 mm, 8 mm, 10 mm, 12 mm, 15 mm, 20 mm, 25 mm, 30 mm, 35 mm, 40 mm, 45 mm 50 mm, 55 mm, 60 mm, 65 mm, up to about 70 mm. A half inch (about 12 mm) mesh size can be used to contain fingerlings.

An array of spaced rotary panels is disposed between the inner cage and the outer cage and serves as a means for regulating flow of matter between the cages. The array can be disposed within the confines of or outside the perimeter of the inner cage. Preferably, the array is disposed immediately next to the meshed side walls. In one embodiment, the array of spaced rotary panels is a part of the inner cage. The rotary panels are dimensioned and profiled according to the size and shape of the inner cage, and are aligned with the meshed side walls. The array of panels regulate the flow of fluids in and out of the inner cage by increasing or decreasing, or opening or closing gaps between the spaced panels. The rotary panels, elongate and substantially rectangular in shape, can be disposed in a vertical or horizontal position. The panel may comprise a shaft that is embedded along the entire length of the panel or coupled to one of its longitudinal edge. The panel may comprise one or more short rods at the end(s) of the panel. The rods and/or shafts facilitate rotation of the panel, and secure the panel to a frame. In various embodiments, the panels function like louvers to regulate the lateral flow of fluid through gaps between the panels.

In one embodiment, the rotary panels are held in a vertical position, and can each rotate upon itself around a vertical, longitudinal axis that lies substantially parallel to the side walls of the inner cage, or substantially perpendicular to the surface of the water. The axis can be located at any point along the width of the panel and can coincide with the rod(s) or shaft if they are present. Preferably, the panels are equidistantly-spaced along the perimeter of the inner cage or a segment thereof. Preferably, the array is annular and aligned with the interior face of the meshed side walls.

In another embodiment, the rotary panels are held in a horizontal position, and can each rotate upon itself around a horizontal axis that is substantially parallel to the surface of the water. The axis can be located at any point along the width of the panel and can coincide with the rod(s) or shaft if they are present. Preferably, the panels are equidistantly-spaced from the top to the bottom of the array. Preferably, the array is in the shape of a polygon with n sides, and n sets of horizontal panels are disposed one on each side of the polygon, wherein n is an integer, such as 4, 5, 6, 7, 8, 10, 12, 15, 20 or more. The polygon can be a regular polygon, e.g., a square, a pentagon, a hexagon, a heptagon, an octagon, etc.

In preferred embodiments, the width (where the panels are held vertically) or height (where the panels are held horizontally) of the panels are equal to or greater than the gap between two neighboring panels. The gap can be measured from the rotational axes of two neighboring panels. Panels that are wider than the gap overlap the next panel when they are in a closed position, thereby creating a more effective barrier to fluid flow. The rotation of the panels in an array is preferably controlled coordinately such that the direction and angle of rotation of the panels are uniform. For example, the panels in an array can be connected via a sprocket and chain mechanism to a motor which rotates the panels in unison. The sprocket can be attached to the panel, rods, or shaft.

By rotating and holding the panels in the same direction at about the same angle, the inner cage can be converted from an open cage to a closed cage, and vice versa. The lateral flow of fluid in and out of the inner cage is controlled by adjusting the angular position of the rotary panels relative to the side walls of the inner cage. For vertically-held panels, the angular position is observed from the top. For horizontally-held panels, the angular position is observed from the sides. To reduce or stop the lateral flow of fluid through the gaps between panels, a panel is rotated so that the angle formed between the face of the panel and its nearest meshed side wall is acute and minimized, or that is approaching about 0°. A rotary panel is in a closed position when the face of the panel is parallel to the meshed side wall, and thus creating a barrier next to the meshed side wall. In certain embodiments, due to the width of the panels, the panels cannot rotate to a position that is completely parallel to the side wall because the outer edge of a panel contacts the face of their neighboring panel, thereby closing any gaps between the panels. To increase the flow of fluid, a panel is rotated so that the angle between the panel and its nearest side wall increases and when it approaches a right angle, about 90° or perpendicular, the inner cage is considered to be in a fully open position. The rotary panel is in a fully closed position when the angular position approaches 0°. Any intermediate angular position between 0° to 90°, such as but not limited to 5°, 10°, 20°, 30°, 40°, 45°, 50°, 60°, 70° and 80° would allow a certain amount of fluid to flow between the panels.

Each rotary panel can be, independently, flat, concave, or convex. The rotary panels are formed with a continuous material that is impermeable to solid waste produced by the fishes in the inner cage, or impermeable to water. The continuous material can be plastics, such as but not limited to vinyl or neoprene, which are impermeable to water, or a closed fabric made with weaved natural or artificial fibers, such as canvas, which are permeable to fluids but not the solid waste. In certain embodiments, one or more of the edges of the panels are tapered and resemble blades.

The bottom of the cage can be made of any continuous material, e.g., plastic or closed fabric. This continuous material is stretched horizontally across the bottom of the cage, and it preferably rises vertically along the perimeter to a height equal to or higher than the base of the rotary panels. This is to prevent the formation of an unintentional gap between the rotary panels in the closed position and the bottom which may result in outward flow of suspended solids and settled solids to the outer cage due to water motions and centripetal forces. A meshed aperture in the bottom of the inner cage, preferably centrally disposed, is provided to allow solid wastes to be directed to the planktivores/herbivores containing cage or the detritivores containing cage. The planktivores/herbivores containing outer cage, can comprise conventional meshed side walls, or side walls that include a series of panels disposed around its perimeter, like the inner cage. In another embodiment, an optional third cage can be placed directly beneath or proximate to the inner and/or outer cages. In yet another embodiment, a receptacle is connected to the aperture of the inner cage or the bottom of the inner and outer cages, to receive and accumulate solid wastes which may be conveyed via a duct by a submersible pump, e.g., a sludge pump to the detritivores in a third cage or to a surface-based receiver, e.g., a tank on a service boat. The solid wastes are then transported to a land-based disposal site, such as a landfill or a compost pit.

The side walls of a cage, and the array of panels provided herein are, independently or integrally, fastened to, mounted onto or suspended from, a frame which has a polygonal or circular cross-section and comprises a plurality of supporting frame members. The supporting frame member can be located at the vertices of a polygonal frame. Alternatively, the cage and/or the array can be constructed with a central spar and radial frame members. A mechanism for rotating the panels, preferably in unison, such as sprockets and a chain, can be attached to the frame. In certain embodiments, the top and bottom of the frame can each comprise a buoyant frame member. The perimeter of top frame member can be different from the perimeter of the bottom frame member, resulting in a frusto-polyhedral shaped cage. In a preferred embodiment, the perimeter of the top frame member is greater than that of the bottom frame member, resulting in a funnel-shaped cage. The funnel shape assists the passage of solid materials from the inner cage to the outer cage through the meshed aperture on the bottom. In a preferred embodiment, the two cages are nested, floating, cylindrical cages that are coaxially disposed.

A means for producing a unidirectional current is provided in the inner cage. The means can be a single function device that generates the current, such as but not limited to a water pump, a propeller, or an agitator; or a combination device that generates the current and also aerate the water in the cage, such as but not limited to a venturi aerator, an air lift, or an air pump. A non-limiting example is a columnar high pressure manifold for producing water and/or air jet flow. Such a device can also be used for sequential, simultaneous, or periodic diffusion of oxygen gas or oxygen-saturated water in the inner cage. The device can comprise one or more rows of nozzles that lies along the longitudinal axis of the device and that points in the same direction.

The water current produced by the device(s) can facilitate a net flow of water from the inner cage to the outer cage. The rotary panels of the inner cage are rotated, preferably in unison, to the open position or other intermediate positions to allow the outflow. The outward flow of water carries dissolved nutrients and fine particulate wastes through gaps between the panels and through the meshed side walls. Preferably, the cages are operating in this mode when heavier particulates that can settle to the bottom are settled on the bottom or removed from the inner cage.

The water current produced by the device(s) can also form a circular and/or a spiraling motion of the water in the inner cage. Several devices can be used advantageously in the inner cage each positioned and configured to generate a current that flow around the cage in the same direction. The circular and/or spiraling motion of the water in the inner cage directs denser settle-able solids or settled solid wastes, such as uneaten food and feces, towards the lower section of the inner cage where they settle on the bottom of the inner cage and flow toward the meshed aperture on the bottom of the inner cage where the wastes exit the inner cage. The circular and/or spiraling motion of the water can generate a centripetal force that moves the settling or settled solids towards the center of the cage and the particles accrete together and become larger and heavier leading to an acceleration in settlement. Generally, the panels are rotated to the fully closed position to optimize the circular and/or spiral water current formation inside the inner cage and to prevent escape of such wastes from the inner cage to the outer cage, or from the outer cage to ambient water. The direction of the circular and/or spiral water current inside the inner cage may be clockwise or counterclockwise and is determined by the directionality of one or more devices that generate the current. The rotary panels, especially when held vertically, are preferably rotated so that the outer edges of the panels point in the same direction as the current, so that the current flows over the rotary panels in a smooth fashion. Whereas, if the outer edges of the panels are pointing in a direction that opposes the water current, the panels may open or offer resistance and reduce the velocity of the circular and/or spiral water current. Therefore, angular position and direction of the rotary panels are adjusted to match the directionality of the current-producing device(s) in order to maximize and maintain the water current in the cage.

To compensate for the outward flow of water from the inner cage, an inflow of water is provided. In certain embodiments, the means for producing current draws aerated and clean water from outside of the cages into the inner cage. Depending on the sizes of the cages and water pressure in the current producing devices, a volume that is 5% to 50% of the volume of the inner cage may be exchanged daily. The diameter of the inner cage can range from about 10 feet to about 200 feet, preferably from about 50 feet to 100 feet. Generally, the diameter of the outer cage is at least two to five times the diameter of the inner cage. The size of the outer cage is in part governed by the intended operation of the system. When operating in eutrophic water, to facilitate the uptake of allochthonous nutrients, the diameter of the outer cage is more than two times as the rate of nutrient loading is orders of magnitude higher than from formulated fish feeds given to the fish in the inner cage when operating as a commercial fish farming system. The second cage or outer cage can also comprise a second array of spaced rotary panels, each being rotatable about a vertical or horizontal axis to increase or decrease, open or close, gaps between neighboring panels which regulate the flow of water from the second or outer cage to ambient water.

In certain embodiments, the cages float independently in the water with the top of the cages located just above the water surface. Such cages do not require a top cover but optionally they can be provided with a top cover to prevent escape of the organisms, and intrusion or access by predatory organisms, such as crustaceans, mammals (e.g., seal), and birds. For submersible cages, a top cover, preferably meshed, is required. The buoyancy of the frame members can each be adjusted independently to control the vertical mooring of the cages. For example, the cage can be submerged through the flooding of the ballastable frame members. Submersible cages can be kept at surface during calm weather and are lowered into the water during storms.

Each of the cages can comprise one or more gates or closeable portals for the fish to enter or leave the respective cages. The gates or portals can be located on any walls, top or bottom of cage, or integrally built into one or more frame members. In certain embodiments, a gate/portal allows the fishes from the inner cage to enter the outer cage. In other embodiments, a gate/portal allows the fishes from the outer cage to enter the inner cage. The gates and portals can be used to allow the piscivorous fishes to feed on fishes in the outer cage and/or bottom cage.

Techniques for shellfish culture are well known in the art and can be adapted to be used in the systems provided herein without undue experimentation. For example, shellfishes that have been attached to ropes can be suspended from frame members that form the top of the cages. Rack culture can be accomplished by adapting horizontal and traverse frame members inside a cage into racks. Ropes, sticks, or nylon mesh bags with shellfishes attached or contained are placed on the racks for growout.

The cages are constructed to have a surface area and depth that allow exposure of the algae to light that sustains algal growth. The algae that are added to or preexisting in the cages, grow in the cages, and are consumed by the fishes and/or shellfishes in the outer cages. In a specific embodiment, the algae can be added to, pumped into, or allowed to flow into the planktivores/herbivores containing cage. The water in the cages may be fertilized regularly according to conventional fishery practices. Vitamins, such as thiamin, riboflavin, pyroxidine, folic acid, panthothenic acid, biotin, inositol, choline, niacin, Vitamin B12, vitamin C, vitamin A, vitamin D, vitamin E, vitamin K; and minerals, such as but not limited to calcium, phosphorous, magnesium, iron, copper, zinc, manganese, iodine and selenium, required for optimal fish growth, and other aquaculture additives, such as antibiotics, may be provided. In certain embodiments, the planktivorous, herbivorous, and/or detritivorous fishes are the only major source of food that provide energy and support growth of the piscivorous fishes. The transfer of fishes between cages can be affected by allowing the fishes to swim from one cage to another cage.

The systems provided herein can operate within the following non-limiting, exemplary water quality limits: dissolved oxygen at greater than 5 mg/L, pH 6-10 and preferably pH from 6.5-8.2 for cold water fishes and pH 7.5 to 9.0 for warm water fishes; alkalinity at 10-400 mg/L CaCO₃; salinity at 0.1-3.0 g/L for stenohaline fishes and 28-35 g/L for marine fishes; less than 0.5 mg ammonia/L; less than 0.2 mg nitrite/L; and less than 10 mg/L CO₂. Techniques and equipments commonly employed in the aquaculture industry can be used for monitoring the aquatic environments of the system. See, for example, the instrumentation and monitoring technology described in Chapter 19 of Aquaculture Engineering, Odd-Ivar Lekang, 2007, Blackwell Publishing Ltd.

The cages and operating subsystems can be set up according to knowledge known in the art, see, for example, Chapters 13 in Aquaculture Engineering, Odd-Ivar Lekang, 2007, Blackwell Publishing Ltd.; and Chapters 3 and 7, Cage Aquaculture. M. Beveridge, third edition, 2004, Blackwell Publishing Ltd. Feeding subsystem and means for feeding fish in culture can be used or adapted if necessary to control the feeding of algae to the fishes, see e.g., Chapter 16, Aquaculture Engineering, Odd-Ivar Lekang, 2007; Chapter 6, Cage Aquaculture, M. Beveridge, third edition, 2004. Various techniques and means for oxygenation of water known in the art can be applied in the method provided herein, see, for example, Chapter 8 in Aquaculture Engineering, Odd-Ivar Lekang, 2007. Conventional fish hatcheries and farming techniques known in the art can be applied to implement the system and methods provided herein, see for example, Chapters 10, 13, 15 in Aquaculture Engineering, Odd-Ivar Lekang, 2007.

### 5.2 Fishes

As used herein, the term fish refers to a member or a group of the following classes: Actinopteryii (i.e., ray-finned fish) which includes the division Teleosteri (also known as the teleosts), Chondrichytes (e.g., cartilaginous fish), Myxini (e.g., hagfish), Cephalospidomorphi (e.g., lampreys), and Sarcopteryii (e.g., coelacanths). The teleosts comprise at least 38 orders, 426 families, and 4064 genera. Some teleost families are large, such as Cyprimidae, Gobiidae, Cichlidae, Characidae, Loricariidae, Balitoridae, Serranidae, Labridae, and Scorpaenidae. In many embodiments, provided herein are bony fishes, such as the teleosts, and/or cartilaginous fishes. When referring to a plurality of organisms, the term “fish” is used interchangeably with the term “fishes” regardless of whether one or more than one species are present, unless clearly indicated otherwise. Stocks of fish used in certain embodiments can be obtained initially from fish hatcheries or collected from the wild. Preferably, cultured or farmed fishes are used in certain embodiments. The fishes may be fish fry, juveniles, fingerlings, or adult/mature fish. In certain embodiments, fry and/or juveniles that have metamorphosed are used. By “fry” it is meant a recently hatched fish that has fully absorbed its yolk sac, while by “juvenile” or “fingerling,” it is meant a fish that has not recently hatched but is not yet an adult.

Fish inhabits most types of aquatic environment, including but not limited to freshwater, brackish, marine, and briny environments. As the present systems and methods can be practiced in any of such aquatic environments, any freshwater species, stenohaline species, euryhaline species, marine species, species that grow in brine, and/or species that thrive in varying and/or intermediate salinities, can be used. Depending on the latitude of the system, fishes from tropical, subtropical, temperate, polar, and/or other climatic regions can be used. For example, fishes that live within the following temperature ranges can be used: below 10° C., 9° C. to 18° C., 15° C. to 25° C., 20° C. to 32° C. In one embodiment, fishes indigenous to the region at which the caged aquaculture methods provided herein are practiced, are used. The algae and the fishes are preferably derived from a naturally occurring trophic system.

In an aquatic ecosystem, fish occupies various trophic levels. Depending on diet, fish are classified generally as piscivores (carnivores), herbivores, planktivores, detritivores, and omnivores. The classification is based on observing the major types of food consumed by fish and its related adaptation to the diet. For example, many species of planktivores develop specialized anatomical structures to enable filter feeding, e.g., gill rakers and gill lamellae. Generally, the size of such filtering structures relative to the dimensions of plankton, including microalgae, affects the diet of a planktivore. Fish having more closing spaced gill rakers with specialized secondary structures to form a sieve are typically phytoplanktivores. Others having widely spaced gill rakers with secondary barbs are generally zooplanktivores. In the case of piscivores, the gill rakers are generally reduced to barbs. Herbivores generally feed on macroalgae and other aquatic vascular plants. Gut content analysis can determine the diet of a fish used in certain embodiments. Techniques for analysis of gut content of fish are known in the art. As used herein, a planktivore is a phytoplanktivore if a population of the planktivore, reared in water with non-limiting quantities of phytoplankton and zooplankton, has on average more phytoplankton than zooplankton in the gut. Under similar conditions, a planktivore is a zooplantivore if the population of the planktivore has on average more zooplankton than phytoplankton in the gut.

In various embodiments, the population of fish in the inner cage comprises predominantly piscivores or carnivores, or fishes that are fed with formulated or pelletized aquaculture feed in conventional fish farms. These types of fishes are cultured in a cage separately from the planktivores, herbivores, and detritivores described above. In various embodiments, the population of fish in the outer cage comprises predominantly planktivores. In certain embodiments, one or several major species in the outer cage are herbivores. In certain embodiments, one or several major species in the outer cage are phytoplanktivores. In other embodiments, one or several species in the outer cage are zooplanktivores. In certain embodiments, one or several major species in the bottom cage are detritivores. In some embodiments, the population of fish in one of the cages comprises predominantly omnivores. In certain mixed fish population provided herein, planktivores, herbivores, detritivores, and omnivores are present in the outer cage.

Depending on the local environment and the species of fish used, the fish can be introduced at various density from about 50 to 100, about 100 to 300, about 300 to 600, about 600 to 900, about 900 to 1200, and about 1200 to 1500 individuals per m². For common carp, tilapia, and catfish, the minimum stocking density is about 80 fish/m³. The maximum stock density can be estimated from the number of fish that will collectively weigh about 150 kg/m³ when the fish reach a predetermined harvest size. If the harvest weight of the fish is 500 g, for a cage of one cubic meter, 300 fish are stocked The size of fingerling used in stocking the cages is about 15 g. A 15-g fish will be retained by a 13-mm bar mesh net. Larger fish can also be stocked into the cages. Survival rates in well-placed and well-managed cages are typically 98 to 100%. The carrying capacity of a body of water limits the weight of fish that can be cultured. Overstocking beyond the carrying capacity will result in increased stress, disease, mortality, reduced feed conversion efficiency, growth rate, and profit. When cages are set in closed ponds or small closed water bodies, generally, 1,000 m² of water surface area is required to support 400 kg of fish so as not to exceed the carrying capacity of the pond. Whereas in fluvial waters, open coastal waters and offshore areas, the quantity of fish per meter cube of water is determined by the water exchange or flushing rate through the cage and the minimum space the fish species requires to sustain optimal health and growth. A person of skill in the art can determine the number of fish which can be stocked into a cage to assure that the weight does not reach the carrying capacity of the water body during culture.

In certain embodiments, the population of fish in a cage comprises only one species of fish. In another embodiment, the fish population in a cage is mixed and thus comprises one or several major species of fish. A major species is one that ranks high in the head count, e.g., the top one to five species with the highest head count relative to other species. The one or several major fish species may constitute greater than about 10%, about 20%, about 30%, about 40%, about 50%, about 60%, about 70%, about 75%, about 80%, about 90%, about 95%, about 97%, about 98% of the fish present in the population. In certain embodiments, several major fish species may each constitute greater than about 10%, about 20%, about 30%, about 40%, about 50%, about 60%, about 70%, or about 80% of the fish present in the population. In various embodiments, one, two, three, four, five major species of fish are present in a population. Accordingly, a mixed fish population or culture can be described and distinguished from other populations or cultures by the major species of fish present. The population or culture can be further described by the percentages of the major and minor species, or the percentages of each of the major species. It is to be understood that mixed cultures having the same genus or species may be different by virtue of the relative abundance of the various genus and/or species present.

Fishes from different taxonomic groups can be used in the different cages provided herein. It should be understood that, in various embodiments, fishes within a taxonomic group, such as a family or a genus, can be used interchangeably in various methods provided herein. The invention is described below using common names of fish groups and fishes, as well as the scientific names of exemplary species. Databases, such as FishBase by Froese, R. and D. Pauly (Ed.), World Wide Web electronic publication, www.fishbase.org, version (06/2008), provide additional useful fish species within each of the taxonomic groups that are useful in certain embodiments. It is contemplated that one of ordinary skill in art could, consistent with the scope of certain embodiments provided herein, use the databases to specify other species within each of the described taxonomic groups for use in the methods provided herein.

In certain embodiments, the fish population comprises fishes in the order Acipeneriformes, such as but not limited to, sturgeons (trophic level 3) e.g., Acipenser species, Huso huso, and paddlefishes (plankton-feeder), e.g., Psephurus gladius, Polyodon spathula, and Pseudamia zonata.

In certain embodiments, the fish population comprises fishes in the order Clupiformes which include the following families: Chirocentridae, Clupeidae (menhadens, shads, herrings, sardines, hilsa), Denticipitidae, Engraulidae (anchovies). Exemplary members within the order Clupiformes include but not limited to, the menhadens (Brevoortia species), e.g, Ethmidium maculatum, Brevoortia aurea, Brevoortia gunteri, Brevoortia smithi, Brevoortia pectinata, Gulf menhaden (Brevoortia patronus), and Atlantic menhaden (Brevoortia tyrannus); the shads, e.g., Alosa alosa, Alosa alabamae, Alosa fallax, Alosa mediocris, Alosa sapidissima, Alosa mediocris, Dorosoma petenense; the herrings, e.g., Etrumeus teres, Harengula thrissina, Pacific herring (Clupea pallasii pallasii), Alosa aestivalis, Ilisha africana, Ilisha elongata, Ilisha megaloptera, Ilisha melastoma, Ilisha pristigastroides, Pellona ditchela, Opisthopterus tardoore, Nematalosa come, Alosa aestivalis, Alosa chrysochloris, freshwater herring (Alosa pseudoharengus), Arripis georgianus, Alosa chrysochloris, Opisthonema libertate, Opisthonema oglinum, Atlantic herring (Clupea harengus), Baltic herring (Clupea harengus membras); the sardines, e.g., Ilisha species, Sardinella species, Amblygaster species, Opisthopterus equatorialis, Sardinella aurita, Pacific sardine (Sardinops sagax), Harengula clupeola, Harengula humeralis, Harengula thrissina, Harengula jaguana, Sardinella albella, Sardinella janeiro, Sardinella fimbriata, oil sardine (Sardinella longiceps), and European pilchard (Sardina pilchardus); the hilsas, e.g., Tenuolosa species, and the anchovies, e.g., Anchoa species, Engraulis species, Thryssa species, anchoveta (Engraulis ringens), European anchovy (Engraulis encrasicolus), Australian anchovy (Engraulis australis), and Setipinna phasa, Coilia dussumieri.

In certain embodiments, the fish population comprises fishes in the superorder Ostariophysi which include the order Gonorynchiformes, order Siluriformes, and order Cypriniformes. Non-limiting examples of fishes in this group include milkfishes, catfishes, barbs, carps, danios, goldfishes, loaches, shiners, minnows, and rasboras. Milkfishes, such as Chanos chanos, are plankton feeders. The catfishes, such as channel catfish (Ictalurus punctatus), blue catfish (Ictalurus furcatus), catfish hybrid (Clarias macrocephalus), Ictalurus pricei, Pylodictis olivaris, Brachyplatystoma vaillantii, Pinirampus pirinampu, Pseudoplatystoma tigrinum, Zungaro zungaro, Platynematichthys notatus, Ameiurus catus, Ameiurus melas are detritivores. Carps are freshwater herbivores, plankton and detritus feeders, e.g., common carp (Cyprinus carpio), Chinese carp (Cirrhinus chinensis), black carp (Mylopharyngodon piceus), silver carp (Hypophthalmichthys molitrix), bighead carp (Aristichthys nobilis) and grass carp (Ctenopharyngodon idella). Shiners includes members of Luxilus, Cyprinella and Notropis genus, such as but not limited to, Luxilus cornutus, Notropis jemezanus, Cyprinella callistia. Other useful herbivores, plankton and detritus feeders are members of the Labeo genus, such as but not limited to, Labeo angra, Labeo ariza, Labeo bata, Labeo boga, Labeo boggut, Labeo porcellus, Labeo kawrus, Labeo potail, Labeo calbasu, Labeo gonius, Labeo pangusia, and Labeo caeruleus.

In certain embodiments, the fish population comprises fishes in the superorder Protacanthopterygii which include the order Salmoniformes and order Osmeriformes. Non-limiting examples of fishes in this group include the salmons, e.g., Oncorhynchus species, Salmo species, Arripis species, Brycon species, Eleutheronema tetradactylum, Atlantic salmon (Salmo salar), red salmon (Oncorhynchus nerka), and Coho salmon (Oncorhynchus kisutch); and the trouts, e.g., Oncorhynchus species, Salvelinus species, Cynoscion species, cutthroat trout (Oncorhynchus clarkii), and rainbow trout (Oncorhynchus mykiss); which are trophic level 3 carnivorous fish. Other non-limiting examples include the smelts and galaxiids (Galaxia speceis). Smelts are planktivores, for example, Spirinchus species, Osmerus species, Hypomesus species, Bathylagus species, Retropinna retropinna, and European smelt (Osmerus eperlanus).

In certain embodiments, the fish population comprises fishes in the superorder Acanthopterygii which include the order Mugiliformes, Pleuronectiformes, and Perciformes. Non-limiting examples of this group are the mullets, e.g., striped grey mullet (Mugil cephalus), which include plankton feeders, detritus feeders and benthic algae feeders; flatfishes which are carnivorous; the anabantids; the centrarchids (e.g., bass and sunfish); the cichlids, the gobies, the gouramis, mackerels, perches, scats, whiting, snappers, groupers, barramundi, drums wrasses, and tilapias (Oreochromis sp.). Examples of tilapias include but are not limited to nile tilapia (Oreochromis niloticus), red tilapia (O. mossambicus×O. urolepis hornorum), mango tilapia (Sarotherodon galilaeus).

Non-limiting examples of species for culturing in the inner cages of freshwater systems, include fishes that are consumers of pelletized aquaculture feeds such as tilapias (Oreochromis sp.) and its hybrids (e.g, red tilapia (Oreochromis mossambica) and Black tilapia (Oreochromis niloticus); European, Asian and Chinese carps (e.g., Cyprinus carpio, Catla catla, Hypophthalmichthys nobilis), catfish, sturgeons (e.g., Ascipenser transmontanus); and carnivores, many of which are in the order Perciformes, such as large mouth bass (Micropterus salmoides), hybrid striped bass (e.g., a cross between striped bass Morone saxatilis and White bass M. chrysops), and Nile perch (Lates niloticus). Non-limiting examples of species for culturing in the outer cages of freshwater systems, include species that are generally of the order Clupeiformes, e.g., native American Shad (Alosa sapidissima), Atheriniformes, e.g., native inland Silverside (Menidia beryllina); Cypriniformes, e.g., native Texas shiner (Notropis amabilis). Non-limiting examples of freshwater detritivores species for inner, outer, and/or third cage are generally of the Order Siluriformes e.g., native channel catfish (Ictalurus punctatus); Order Characiformes, e.g., Streaked prochilod (Prochilodus lineatus); order Mugiliformes, e.g., native mountain mullet (Agonostomus monticola), and may also include native Freshwater drum (Aplodinotus grunniens), and native Mrigal (Cirrhinus cirrhosus).

Non-limiting examples of species for culturing in the inner cages of marine systems, include Perciformes such as red snapper (Lutjanus campechanus), snook (Centropomus undecimalis), white sturgeon (Acipenser transmontaus), Pompano, (Trachinotus carolinus), red grouper (Epinephelus morio, red drum (Sciaenops ocellantus), and barramundi (Lates calcarifer). Non-limiting examples of species for culturing in the outer cages of marine systems include Clupieforms such as Atlantic herring (Chupea harengus harengus), gulf menhaden (Brevoortia patronus), Northern anchovy (Engraulis mordax). Non-limiting examples of detritivores for marine systems include marine catfish, mullets, and black drum.

The shellfishes used in the methods provided herein are preferably sedentary shellfishes, such as bivalves. Depending on the environment, freshwater, brackish water, or marine shellfishes can be used. The shellfishes provided herein include but are not limited to oysters, mussels, scallops, clams, and more particularly, Crassostrea species such as C. gigas, C. virginica, C. ariakensis, C. rivularis, C. angulata, C. eradelie, C. commercialis, Saccostrea species such as S. glomerata, S. cucculata and S. commercialis, Mercenaria species such as M. mercenaria and M. campechensis, Ostrea species such as O. edulis, O. chilensis, and O. lurida, Area transversa, Panope generosa, Saxodomus nuttili, Mytilus species such as M. edulis (blue mussel), M. coruscus, M. chilensis, M. trossulus, and M. galloprovincialis (Mediterranean mussel), Aulacomya ater, Choromytilus chorus, Tapes semidecussatus, Perna species such as P. viridis, P. canaliculus, Venerupis species such as V. decussata, V. semidecussata, Sinonovacula constricta (Razor clam), Mya arenaria (soft shell clams), Spisula solidissima (surf clams), Amusium balloti, Argopecten irradians (bay scallops), Pectan species such as P. alba, P. yessonsis, P. maximus, and Chlamys species such as C. farreri, C. opercularis, C. purpuratus and C. varia.

The fishes can be gathered or harvested by any methods or means known in the art. In some embodiments, a fish gathering or capturing means is configured to separate fish based on a selected physical characteristic, such as density, weight, length, or size. The means to gather or capture fish can be mechanical, pneumatic, hydraulic, electrical, or a combination of various mechanisms, e.g., nets. For example, see Chapters 17 and 19 in Aquaculture Engineering, Odd-Ivar Lekang, 2007, Blackwell Publishing Ltd., for description of techniques and means for moving and grading fish. Harvested fishes can be sold as human food fish in live fish markets. Harvested fish can also be processed for value added products such as fillets, or processed for high quality fish oil and fish protein concentrate for human consumption. Harvested fish including fish viscera can also be rendered to produce fish meal and fish oil for use in animal or fish feeds, or processed to make biofuels or industrial feedstock.

In certain embodiments, provided herein are a biofuel feedstock or a biofuel comprising lipids, hydrocarbons, or both, derived from fish that harvested algae according to the methods provided herein. Lipids obtained by the systems and methods provided herein can be subdivided according to polarity: neutral lipids and polar lipids. The major neutral lipids are triglycerides, and free saturated and unsaturated fatty acids. The major polar lipids are acyl lipids, such as glycolipids and phospholipids. A composition comprising lipids and hydrocarbons obtained by the systems and methods provided herein can be described and distinguished by the types and relative amounts of key fatty acids and/or hydrocarbons present in the composition.

Fatty acids are identified herein by a first number that indicates the number of carbon atoms, and a second number that is the number of double bonds, with the option of indicating the position of the first double bond or the double bonds in parenthesis. The carboxylic group is carbon atom 1 and the position of the double bond is specified by the lower numbered carbon atom. For example, linoleic acid can be identified by 18:2 (9, 12).

Algae produce mostly even-numbered straight chain saturated fatty acids (e.g., 12:0, 14:0, 16:0, 18:0, 20:0 and 22:0) with smaller amounts of odd-numbered acids (e.g., 13:0, 15:0, 17:0, 19:0, and 21:0), and some branched chain (iso- and anteiso-) fatty acids. A great variety of unsaturated or polyunsaturated fatty acids are produced by algae, mostly with C₁₂ to C₂₂ carbon chains and 1 to 6 double bonds, mainly in cis configurations. Fatty acids produced by the cultured algae provided herein comprise one or more of the following: 12:0, 14:0, 14:1, 15:0, 16:0, 16:1, 16:2, 16:3, 16:4, 17:0, 18:0, 18:1, 18:2, 18:3, 18:4, 19:0, 20:0, 20:1, 20:2, 20:3, 20:4, 20:5, 22:0, 22:5, 22:6, and 28:1 and in particular, 18:1(9), 18:2(9, 12), 18:3(6, 9, 12), 18:3(9, 12, 15), 18:4(6, 9, 12, 15), 18:5(3, 6, 9, 12, 15), 20:3(8, 11, 14), 20:4(5, 8, 11, 14), 20:5(5, 8, 11, 14, 17), 20:5(4, 7, 10, 13, 16), 20:5(7, 10, 13, 16, 19), 22:5(7, 10, 13, 16, 19), 22:6(4, 7, 10, 13, 16, 19). Without limitation, it is expected that many of these fatty acids are present in the lipids extracted from the fishes that ingested the cultured algae.

The hydrocarbons present in algae are mostly straight chain alkanes and alkenes, and may include paraffins and the like having up to 36 carbon atoms. The hydrocarbons are identified by the same system of naming carbon atoms and double bonds as described above for fatty acids. Non-limiting examples of the hydrocarbons are 8:0, 9,0, 10:0, 11:0, 12:0, 13:0, 14:0, 15:0, 15:1, 15:2, 17:0, 18:0, 19:0, 20:0, 21:0, 21:6, 23:0, 24:0, 27:0, 27:2(1, 18), 29:0, 29:2(1, 20), 31:2(1, 22), 34:1, and 36:0.

A great variety of unsaturated or polyunsaturated fatty acids are produced by fish mostly with C₁₂ to C₂₂ carbon chains and 1 to 6 double bonds, mainly in cis configurations (Stansby, M. E., “Fish oils,” The Avi Publishing Company, Westport, Conn., 1967). Fish oil comprises about 90% triglycerides, about 5-10% monoglycerides and diglycerides, and about 1-2% sterols, glyceryl ethers, hydrocarbons, and fatty alcohols. One of skill would understand that the amount and variety of lipids in fish oil varies from one fish species to another, and also with the season of the year, the algae diet, spawning state, and environmental conditions. Fatty acids produced by the fishes provided herein comprise, without limitation, one or more of the following: 12:0, 14:0, 14:1, 15:branched, 15:0, 16:0, 16:1, 16:2 n-7, 16:2 n-4, 16:3 n-4, 16:3 n-3, 16:4 n-4, 16:4 n-1, 17:branched, 17:0, 17:1, 18:branched, 18:0, 18:1, 18:2 n-9, 18:2 n-6, 18:2 n-4, 18:3 n-6, 18:3 n-6, 18:3 n-3, 18:4 n-3, 19:branched, 19:0, 19:1, 20:0, 20:1, 20:2 n-9, 20:2 n-6, 20:3 n-6, 20:3 n-3, 20:4 n-6, 20:4 n-3, 20:5 n-3, 21:0, 21:5 n-2, 22:0, 22:1 n-11, 22:2, 22:3 n-3, 22:4 n-3, 22:5 n-3, 22:6 n-3, 23:0, 24:0, 24:1 (where n is the first double bond counted from the methyl group). See, also Jean Guillaume, Sadisivam Kaushik, Pierre Bergot, and Robert Metailler, “Nutrition and Feeding of Fish and Crustaceans,” Springer-Praxis, UK, 2001).

In various embodiments, provided herein are methods of making a liquid fuel which comprise processing lipids derived from fish that harvested algae. Products provided herein made by the processing of fish-derived biofuel feedstocks can be incorporated or used in a variety of liquid fuels including but not limited to, diesel, biodiesel, kerosene, jet-fuel, gasoline, JP-1, JP-4, JP-5, JP-6, JP-7, JP-8, Jet Propellant Thermally Stable (JPTS), Fischer-Tropsch liquids, alcohol-based fuels including ethanol-containing transportation fuels, other biomass-based liquid fuels including cellulosic biomass-based transportation fuels.

Triacylglycerides in fish oil can be converted to fatty acid methyl esters (FAME or biodiesel), for example, by using a base-catalyzed transesterification process (for an overview see, e.g., K. Shaine Tyson, Joseph Bozell, Robert Wallace, Eugene Petersen, and Luc Moens, “Biomass Oil Analysis: Research Needs and Recommendations, NREL/TP-510-34796, June 2004). The triacylglycerides are reacted with methanol in the presence of NaOH at 60° C. for 2 hrs to generate a fatty acid methyl ester (biodiesel) and glycerol.

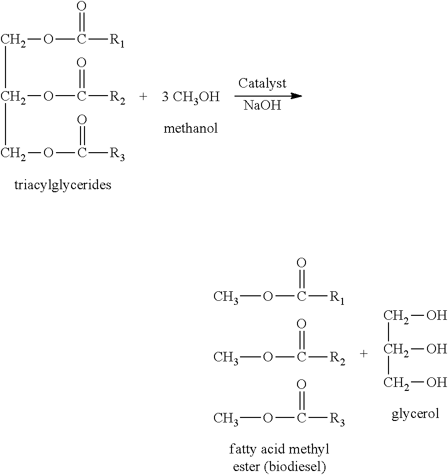

The biodiesel and glycerol co-products are immiscible and typically separated downstream through decanting or centrifugation, followed by washing and purification. Free fatty acids (FFAs) are a natural hydrolysis product of triglyceride and formed by the following reaction with triacylglycerides and water:

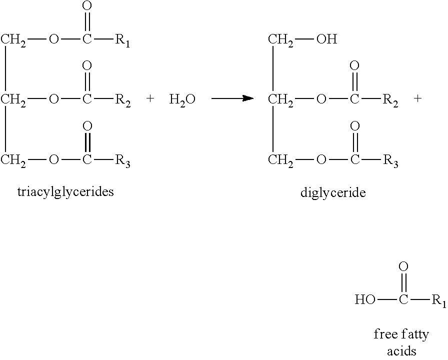

This side reaction is undesirable because free fatty acids convert to soap in the transesterification reaction, which then emulsifies the co-products, glycerol and biodiesel, into a single phase. Separation of this emulsion becomes extremely difficult and time-consuming without additional cost-prohibitive purification steps.

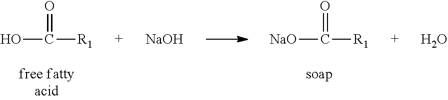

Accordingly, the methods provided herein can further comprise a step for quickly and substantially drying the fish oil by techniques known in the art to limit production of free fatty acids, preferably to less than 1%. In another embodiment, the methods provided herein can further comprise a step for converting or removing the free fatty acids by techniques known in the art.

Triacylglycerides in fish oil can also be converted to fatty acid methyl esters (FAME or biodiesel) by acid-catalyzed transesterification, enzyme-catalyzed transesterification, or supercritical methanol transesterification. Supercritical methanol transesterification does not require a catalyst (Kusdiana, D. and Saka, S., “Effects of water on biodiesel fuel production by supercritical methanol treatment,” Bioresource Technology 91 (2004), 289-295; Kusdiana, D. and Saka, S., “Kinetics of transesterification in rapeseed oil to biodiesel fuel as treated in supercritical methanol,” Fuel 80 (2001), 693-698; Saka, S., and Kusdiana, D., “Biodiesel fuel from rapeseed oil as prepared in supercritical methanol,” Fuel 80 (2001), 225-231). The reaction in supercritical methanol reduces the reaction time from 2 hrs to 5 minutes. In addition, the absence of the base catalyst NaOH greatly simplifies the downstream purification, reduces raw material cost, and eliminates the problem with soaps from free fatty acids. Rather than being a problem, the free fatty acids become valuable feedstocks that are converted to biodiesel in the supercritical methanol as follows.

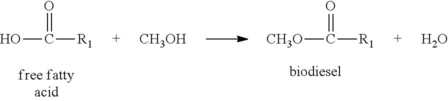

Non-limiting exemplary reaction conditions for both the base-catalyzed and supercritical methanol methods are shown in Table 1 below. As will be apparent to one of ordinary skill in the art, other effective reaction conditions can be applied with routine experimentation to convert the triacylglycerides in fish oil to biodiesel by either one of these methods.

TABLE 1
Comparison between base-catalyzed and supercritical processing

| | Traditional Method | SC Methanol |
|---|---|---|
| Reaction time | 2 hrs | <5 min |
| Conditions | Atmospheric, 60° C. | 1,000 psig, 350° C. |
| Catalyst | NaOH | None |
| FFA product | Soap | Biodiesel |
| Acceptable Water (%) | <1% | No limit |

In another embodiment, triacylglycerides are reduced with hydrogen to produce paraffins, propane, carbon dioxide and water, a product generally known as green diesel. The paraffins can either be isomerized to produce diesel or blended directly with diesel. The primary advantages of hydrogenation over conventional base-catalyzed transesterification are two-fold. First, the hydrogenation process (also referred to as hydrocracking) is thermochemical and therefore much more robust to feed impurities as compared to biochemical processes, i.e., hydrocracking is relatively insensitive to free fatty acids and water. Free fatty acids are readily converted to paraffins, and water simply reduces the overall thermal efficiency of the process but does not significantly alter the chemistry. Second, the paraffin product is a pure hydrocarbon, and therefore indistinguishable from petroleum-based hydrocarbons. Unlike biodiesel which has a 15% lower energy content and can freeze in cold weather, green diesel has similar energy content and flow characteristics (e.g., viscosity) to petroleum-based diesel. In various embodiments, the methods provided herein encompass the steps of hydrocracking and isomerization, which are well known in the art to produce liquid fuels, such as jet-fuel, diesel, kerosene, gasoline, JP-1, JP-4, JP-5, JP-6, JP-7, JP-8, and JPTS.

In yet another embodiment, residual fish biomass, such as fish meal, that remains after the extraction of lipids are used as a feedstock to produce biofuel. Residual fish biomass can be upgraded to bio-oil liquids, a multi-component mixture through fast pyrolysis (for an overview see, e.g., S. Czernik and A. V. Bridgwater, “Overview of Applications of Biomass Fast Pyrolysis Oil,” Energy & Fuels 2004, 18, pp. 590-598; A. V. Bridgwater, “Biomass Fast Pyrolysis,” Thermal Science 2004, 8(8), pp. 21-29); Oasmaa and S. Czernik, “Fuel Oil Quality of Biomass Pyrolysis Oils—State of the Art for End Users,” Energy & Fuels, 1999, 13, 914-921; D. Chiaramonti, A. Oasmaa, and Y. Solantausta, “Power Generation Using Fast Pyrolysis Liquids from Biomass, Renewable and Sustainable Energy Reviews, August 2007, 11(6), pp. 1056-1086). According to certain embodiments provided herein, residual fish biomass is rapidly heated to a temperature of about 500° C., and thermally decomposed to 70-80% liquids and 20-30% char and gases. The liquids, pyrolysis oils, can be upgraded by hydroprocessing to make products, such as naphtha and olefins. Those skilled in the art will know many other suitable reaction conditions, or will be able to ascertain the same by use of routine experimentation.

In yet another embodiment, residual fish biomass can be subjected to gasification which partially oxidizes the biomass in air or oxygen to form a mixture of carbon monoxide and hydrogen or syngas. The syngas can be used for a variety of purposes, such as but not limited to, generation of electricity or heat by burning, Fischer-Tropsch synthesis, and manufacture of organic compounds. For an overview of syngas, see, e.g., Spath, P. L., and Dayton, D. C., “Preliminary Screening—Technical and Ecnomic Assessment of Synthesis Gas to Fuels and Chemicals with Emphasis on the Potential for Biomass-derived Syngas.” NREL/TP-510-34929, December 2003.

In yet another embodiment, residual fish biomass can be subjected to fermentation to convert carbohydrates to ethanol which can be separated using standard techniques. Numerous fungal and bacterial fermentation technologies are known in the art and can be used in accordance with certain embodiments provided herein. For an overview of fermentation, see, e.g., Edgard Gnansounou and Arnaud Dauriat, “Ethanol fuel from biomass: A Review,” Journal of Scientific and Industrial Research, Vol. 64, November 2005, pp 809-821.

In certain embodiments, the processing step involves heating the fishes to greater than about 70° C., 80° C., 90° C. or 100° C., typically by a steam cooker, which coagulates the protein, ruptures the fat deposits and liberates lipids and oil and physico-chemically bound water, and; grinding, pureeing and/or pressing the fish by a continuous press with rotating helical screws. The fishes can be subjected to gentle pressure cooking and pressing which use significantly less energy than that is required to obtain lipids from algae. The coagulate may alternatively be centrifuged. This step removes a large fraction of the liquids (press liquor) from the mass, which comprises an oily phase and an aqueous fraction (stickwater). The separation of press liquor can be carried out by centrifugation after the liquor has been heated to 90° C. to 95° C. Separation of stickwater from oil can be carried out in vertical disc centrifuges. The lipids in the oily phase (fish oil) may be polished by treating with hot water which extracts impurities from the lipids to form biofuel. To obtain fish meal, the separated water is evaporated to form a concentrate (fish solubles) which is combined with the solid residues, and then dried to solid form (presscake). The dried material may be grinded to a desired particle size. The fish meal typically comprises mostly proteins (up to 70%), ash, salt, carbohydrates, and oil (about 5-10%). The fish meal can be used as animal feed and/or as an alternative energy feedstock.

In another embodiment, the fishmeal is subjected to a hydrothermal process that extract residual lipids, both neutral and polar. A large proportion of polar lipids, such as phospholipids, remains with the fish meal and lost as biofuel feedstock. Conversion of such polar lipids into fatty acids can boost the overall yield of biofuel from fish. The hydrothermal process provided herein generally comprises treating fish meal with near-critical or supercritical water under conditions that can extract polar lipids from the fish meal and/or hydrolyze polar lipids resulting in fatty acids. The fish meal need not be dried as the moisture in the fish meal can be used in the process. The process comprises applying pressure to the fish to a predefined pressure and heating the fish meal to a predefined temperature, wherein lipids in the fish meal are extracted and/or hydrolyzed to form fatty acids. The fish meal can be held at one or more of the preselected temperature(s) and preselected pressure(s) for an amount of time that facilitates, and preferably maximizes, hydrolysis and/or extraction of various types of lipids. The term “subcritical” or “near-critical water” refers to water that is pressurized above atmospheric pressure at a temperature between the boiling temperature (100° C. at 1 atm) and critical temperature (374° C.) of water. The term “supercritical water” refers to water above its critical pressure (218 atm) at a temperature above the critical temperature (374° C.). In some embodiments, the predefined pressure is between 5 atm and 500 atm. In some embodiments, the predefined temperature is between 100° C. and 500° C. or between 325° C. and 425° C. The reaction time can range between 5 seconds and 60 minutes. For example, a fish meal can be exposed to a process condition comprising a temperature of about 300° C. at about 80 atm for about 10 minutes. The selection of an appropriate set of process conditions, i.e., combinations of temperature, pressure, and process time can be determined by assaying the quantity and quality of lipids and free fatty acids, e.g., neutral lipids, phospholipids and free fatty acids, that are produced. The process further comprise separating the treated fish meal into an organic phase which includes the lipids and/or fatty acids, an aqueous phase, and a solid phase; and collecting the organic phase as biofuel or feedstock.

The systems provided herein can comprise, independently and optionally, means for gathering fishes from which lipids are extracted (e.g., nets), means for conveying the gathered fishes from the fish enclosure or a holding enclosure to the fish processing facility (e.g., pipes, conveyors, bins, trucks), means for cutting large pieces of fish into small pieces before cooking and pressing (e.g., chopper, hogger), means for heating the fishes to about 70° C., 80° C., 90° C. or 100° C. (e.g., steam cooker); means for grinding, pureeing, and/or pressing the fishes to obtain lipids (e.g., single screw press, twin screw press, with capacity of about 1-20 tons per hour); means for separating lipids from the coagulate (e.g., decanters and/or centrifuges); means for separating the oily phase from the aqueous fraction (e.g., decanters and/or centrifuges); and means for polishing the lipids (e.g., reactor for transesterification or hydrogenation). Many commercially available systems for producing fish meal can be adapted for use in certain embodiments, including stationary and mobile systems that are mounted on a container frame or a flat rack. The fish oil or a composition comprising fish lipids, can be collected and used as a biofuel, or upgraded to biodiesel or other forms of energy feedstock. For example, biodiesel can be produced by transesterification of the fish lipids, and green diesel by hydrogenation, using technology well known to those of skill in the art.

In certain embodiments, the extracted fish lipids are not limited to use as biofuels. In one embodiment, the extracted fish lipids can be used to obtain omega 3 fatty acids, such as eicosahexaenoic acid (EPA) and/or docosahexaenoic acid (DHA) and/or derivatives thereof including, but not limited to esters, glycerides, phospholipids, sterols, and/or mixtures thereof.

In one embodiment, the extracted fish lipids contain substantially purified EPA and/or DHA ranging from 1 to 50%, depending on the fish species, age, location, and a host of ecological and environmental factors. If higher EPA and/or DHA concentrations are desired, several established methods could be employed, including chromatography, fractional or molecular distillation, enzymatic splitting, low-temperature crystallization, supercritical fluid extraction, or urea complexation. These methods can further concentrate the EPA and/or DHA to nearly pure EPA and/or DHA.

In certain embodiments, EPA- and/or DHA-containing lipids may be separated and concentrated by short-path distillation, or molecular distillation. The lipids are first transesterified, either acid- or base-catalyzed, with ethanol to produce a mixture of fatty acid ethyl esters (FAEE). The FAEE are then fractionated in the short-path distillation to remove the short chain FAEE, C-14 to C-18. The concentrate of FAEE from C-20 to C-22 is where the EPA and/or DHA can be found. A second distillation of the concentrate can result in a final Omega-3 content of up to 70%. The concentration of the EPA and/or DHA in the final product will depend on the initial lipid profile of the fish oil. The FAEE can be used as a consumer product at this stage (fish oil capsules). In some countries, the FAEE are required to be reconverted to triglycerides through a glycerolysis reaction before they can be sold as a consumer product. In order to obtain pure EPA and/or DHA, an additional purification step is required using chromatography, enzymatic transesterification, ammonia complexation, or supercritical fluid extraction.

Certain embodiments provide an EPA and/or DHA feedstock or an EPA and/or DHA comprising lipids, hydrocarbons, or both, derived from fish that harvested algae according to the methods provided herein. Lipids of the present embodiments can be subdivided according to polarity: neutral lipids and polar lipids. The major neutral lipids are triglycerides, and free saturated and unsaturated fatty acids. The major polar lipids are acyl lipids, such as glycolipids and phospholipids. A composition comprising lipids and hydrocarbons of the present embodiments can be described and distinguished by the types and relative amounts of key fatty acids and/or hydrocarbons present in the composition.

Fatty acids are identified herein by a first number that indicates the number of carbon atoms, and a second number that is the number of double bonds, with the option of indicating the position of the first double bond or the double bonds in parenthesis. The carboxylic group is carbon atom 1 and the position of the double bond is specified by the lower numbered carbon atom. For example, EPA is identified as 20:5 (n-3), which is all-cis-5,8,11,14,17-eicosapentaenoic acid, and DHA is identified as 22:6 (n-3), which is all-cis-4,7,10,13,16,19-docosahexaenoic acid, or DHA. The n-3 designates the location of the double bond, counting from the end carbon (highest number).

Algae produce mostly even-numbered straight chain saturated fatty acids (e.g., 12:0, 14:0, 16:0, 18:0, 20:0 and 22:0) with smaller amounts of odd-numbered acids (e.g., 13:0, 15:0, 17:0, 19:0, and 21:0), and some branched chain (iso- and anteiso-) fatty acids. A great variety of unsaturated or polyunsaturated fatty acids are produced by algae, mostly with C₁₂ to C₂₂ carbon chains and 1 to 6 double bonds, mainly in cis configurations. Fatty acids produced by the cultured algae of the present embodiments comprise one or more of the following: 12:0, 14:0, 14:1, 15:0, 16:0, 16:1, 16:2, 16:3, 16:4, 17:0, 18:0, 18:1, 18:2, 18:3, 18:4, 19:0, 20:0, 20:1, 20:2, 20:3, 20:4, 20:5, 22:0, 22:5, 22:6, and 28:1 and in particular, 18:1(9), 18:2(9,12), 18:3(6, 9, 12), 18:3(9, 12, 15), 18:4(6, 9, 12, 15), 18:5(3, 6, 9, 12, 15), 20:3(8, 11, 14), 20:4(5, 8, 11, 14), 20:5(5, 8, 11, 14, 17), 20:5(4, 7, 10, 13, 16), 20:5(7, 10, 13, 16, 19), 22:5(7, 10, 13, 16, 19), 22:6(4, 7, 10, 13, 16, 19). Without limitation, it is expected that many of these fatty acids are present in the lipids extracted from the fishes that ingested the cultured algae.

The hydrocarbons present in algae are mostly straight chain alkanes and alkenes, and may include paraffins and the like having up to 36 carbon atoms. The hydrocarbons are identified by the same system of naming carbon atoms and double bonds as described above for fatty acids. Non-limiting examples of the hydrocarbons are 8:0, 9,0, 10:0, 11:0, 12:0, 13:0, 14:0, 15:0, 15:1, 15:2, 17:0, 18:0, 19:0, 20:0, 21:0, 21:6, 23:0, 24:0, 27:0, 27:2(1, 18), 29:0, 29:2(1, 20), 31:2(1,22), 34:1, and 36:0.

A great variety of unsaturated or polyunsaturated fatty acids are produced by fish mostly with C₁₂ to C₂₂ carbon chains and 1 to 6 double bonds, mainly in cis configurations (Stansby, M. E., “Fish oils,” The Avi Publishing Company, Westport, Conn., 1967). Fish oil comprises about 90% triglycerides, about 5-10% monoglycerides and diglycerides, and about 1-2% sterols, glyceryl ethers, hydrocarbons, and fatty alcohols. One of skill would understand that the amount and variety of lipids in fish oil varies from one fish species to another, and also with the season of the year, the algae diet, spawning state, and environmental conditions. Fatty acids produced by the fishes of the present embodiments comprise, without limitation, one or more of the following: 12:0, 14:0, 14:1, 15:branched, 15:0, 16:0, 16:1, 16:2 n-7, 16:2 n-4, 16:3 n-4, 16:3 n-3, 16:4 n-4, 16:4 n-1, 17:branched, 17:0, 17:1, 18:branched, 18:0, 18:1, 18:2 n-9, 18:2 n-6, 18:2 n-4, 18:3 n-6, 18:3 n-6, 18:3 n-3, 18:4 n-3, 19:branched, 19:0, 19:1, 20:0, 20:1, 20:2 n-9, 20:2 n-6, 20:3 n-6, 20:3 n-3, 20:4 n-6, 20:4 n-3, 20:5 n-3, 21:0, 21:5 n-2, 22:0, 22:1 n-11, 22:2, 22:3 n-3, 22:4 n-3, 22:5 n-3, 22:6 n-3, 23:0, 24:0, 24:1 (where n is the first double bond counted from the methyl group). See, also Jean Guillaume, Sadisivam Kaushik, Pierre Bergot, and Robert Metailler, “Nutrition and Feeding of Fish and Crustaceans,” Springer-Praxis, UK, 2001).

In certain embodiments, EPA and/or DHA in the predominant form of triglyceride esters can be converted to lower alkyl esters, such as methyl, ethyl, or propyl esters, by known methods and used in an esterification with a sterol to form esters, which can be further purified for use as nutritional supplement. Transesterification, in general, is well known in the art. See, e.g., W. W. Christie, “Preparation of Ester Derivatives of Fatty Acids for Chromatographic Analysis,” Advances in Lipid Methodology—Volume Two, Ch. 2, pp. 70-82 (W. W. Christie, ed., The Oily Press, Dundee, United Kingdom, 1993).

In certain embodiments, to obtain a refined product with higher concentrations of EPA and/or DHA, certain lipases can be used to selectively transesterify the ester moieties of EPA and/or DHA in fish oil triglycerides, under substantially anhydrous reaction conditions, as described in U.S. Pat. No. 5,945,318.

In certain embodiments, one or more edible additives can be included for consumption with the nutritional supplement of containing EPA and/or DHA. In one embodiment, additives can include one or more antioxidants, such as, vitamin C, vitamin E or rosemary extract. In one embodiment, additives can include one or more suitable dispersant, such as, lecithin, an alkyl polyglycoside, polysorbate 80 or sodium lauryl sulfate. In one embodiment, additives can include a suitable antimicrobial is, for example, sodium sulfite or sodium benzoate. In one embodiment, additives can include one or more suitable solubilizing agent is, such as, a vegetable oil such as sunflower oil, coconut oil, and the like, or mono-, di- or tri-glycerides.

In certain embodiments, additives can include, but not limited to, vitamins such as vitamin A (retinol, retinyl palmitate or retinol acetate), vitamin B1 (thiamin, thiamin hydrochloride or thiamin mononitrate), vitamin B2 (riboflavin), vitamin B3 (niacin, nicotinic acid or niacinamide), vitamin B5 (pantothenic acid, calcium pantothenate, d-panthenol or d-calcium pantothenate), vitamin B6 (pyridoxine, pyridoxal, pyridoxamine or pyridoxine hydrochloride), vitamin B12 (cobalamin or cyanocobalamin), folic acid, folate, folacin, vitamin H (biotin), vitamin C (ascorbic acid, sodium ascorbate, calcium ascorbate or ascorbyl palmitate), vitamin D (cholecalciferol, calciferol or ergocalciferol), vitamin E (d-alpha-tocopherol, or d-alpha tocopheryl acetate) or vitamin K (phylloquinone or phytonadione).

In certain embodiments, additives can include, but not limited to, minerals such as boron (sodium tetraborate decahydrate), calcium (calcium carbonate, calcium caseinate, calcium citrate, calcium gluconate, calcium lactate, calcium phosphate, dibasic calcium phosphate or tribasic calcium phosphate), chromium (GTF chromium from yeast, chromium acetate, chromium chloride, chromium trichloride and chromium picolinate) copper (copper gluconate or copper sulfate), fluorine (fluoride and calcium fluoride), iodine (potassium iodide), iron (ferrous fumarate, ferrous gluconate gluconate, magnesium hydroxide or magnesium oxide), manganese (manganese gluconate and manganese sulfate), molybdenum (sodium molybdate), phosphorus (dibasic calcium phosphate, sodium phosphate), potassium (potassium aspartate, potassium citrate, potassium chloride or potassium gluconate), selenium (sodium selenite or selenium from yeast), silicon (sodium metasilicate), sodium (sodium chloride), strontium, vanadium (vanadium surface) and zinc (zinc acetate, zinc citrate, zinc gluconate or zinc sulfate).

In certain embodiments, additives can include, but not limited to, amino acids, peptides, and related molecules such as alanine, arginine, asparagine, aspartic acid, carnitine, citrulline, cysteine, cystine, dimethylglycine, gamma-aminobutyric acid, glutamic acid, glutamine, glutathione, glycine, histidine, isoleucine, leucine, lysine, methionine, ornithine, phenylalanine, proline, serine, taurine, threonine, tryptophan, tyrosine and valine.

In certain embodiments, additives can include animal extracts such as cod liver oil, marine lipids, shark cartilage, oyster shell, bee pollen and d-glucosamine sulfate. In certain embodiments, additives can include, but not limited to, unsaturated free fatty acids such as .gamma.-linoleic, arachidonic and .alpha.-linolenic acid, which may be in an ester (e.g., ethyl ester or triglyceride) form.

In certain embodiments, additives can include, but not limited to, herbs and plant extracts such as kelp, pectin, Spirulina, fiber, lecithin, wheat genii oil, safflower seed oil, flax seed, evening primrose, borage oil, blackcurrant, pumpkin seed oil, grape extract, grape seed extract, bark extract, pine bark extract, French maritime pine bark extract, muira puama extract, fennel seed extract, dong quai extract, chaste tree berry extract, alfalfa, saw palmetto berry extract, green tea extracts, angelica, catnip, cayenne, comfrey, garlic, ginger, ginseng, goldenseal, juniper berries, licorice, olive oil, parsley, peppermint, rosemary extract, valerian, white willow, yellow dock and yerba mate.

In certain embodiments, additives can include, but not limited to, enzymes such as amylase, protease, lipase and papain as well as miscellaneous substances such as menaquinone, choline (choline bitartrate), inositol, carotenoids (beta-carotene, alpha-carotene, zeaxanthin, cryptoxanthin or lutein), para-aminobenzoic acid, betaine HCl, free omega-3 fatty acids and their esters, thiotic acid (alpha-lipoic acid), 1,2-dithiolane-3-pentanoic acid, 1,2-dithiolane-3-valeric acid, alkyl polyglycosides, polysorbate 80, sodium lauryl sulfate, flavanoids, flavanones, flavones, flavonols, isoflavones, proanthocyanidins, oligomeric proanthocyanidins, vitamin A aldehyde, a mixture of the components of vitamin A₂, the D Vitamins (D₁, D₂, D₃ and D₄) which can be treated as a mixture, ascorbyl palmitate and vitamin K₂.

In certain embodiments, fish meal can be produced from treating fish bodies with a protease acting at a relatively low temperature. In certain embodiments, proteases that can be used include proteinases such as acrosin, urokinase, uropepsin, elastase, enteropeptidase, cathepsin, kallikrein, kininase 2, chymotrypsin, chymopapain, collagenase, streptokinase, subtilisin, thermolysin, trypsin, thrombin, papain, pancreatopeptidase and rennin; peptidases such as aminopeptidases, for example, arginine aminopeptidase, oxytocinase and leucine aminopeptidase; angiotensinase, angiotensin converting enzyme, insulinase, carboxypeptidase, for example, arginine carboxypeptidase, kininase 1 and thyroid peptidase, dipeptidases, for example, carnosinase and prolinase and pronases; as well as other proteases, denatured products thereof and compositions thereof.

Described below are algae that are used in the methods provided herein. Also described are algae present in the body of water that is within the confines of and which percolates the cages provided herein.

As used herein the term “algae” refers to any organisms with chlorophyll and a thallus not differentiated into roots, stems and leaves, and encompasses prokaryotic and eukaryotic organisms that are photoautotrophic or photoauxotrophic. The term “algae” includes macroalgae (commonly known as seaweed) and microalgae. For certain embodiments, algae that are not macroalgae are preferred. The terms “microalgae” and “phytoplankton,” used interchangeably herein, refer to any microscopic algae, photoautotrophic or photoauxotrophic protozoa, photoautotrophic or photoauxotrophic prokaryotes, and cyanobacteria (commonly referred to as blue-green algae and formerly classified as Cyanophyceae). The use of the term “algal” also relates to microalgae and thus encompasses the meaning of “macroalgal.” The term “algal composition” refers to any composition that comprises algae, and is not limited to the body of water or the culture in which the algae are cultivated. An “algal culture” is an algal composition that comprises live algae. The microalgae provided herein are also encompassed by the term “plankton” which includes phytoplankton, zooplankton and bacterioplankton. The methods provided herein can be used in a body of water comprising plankton.

Algae, including microalgae, inhabit all types of aquatic environment, including but not limited to freshwater (less than about 0.5 parts per thousand (ppt) salts), brackish (about 0.5 to about 31 ppt salts), marine (about 31 to about 38 ppt salts), and briny (greater than about 38 ppt salts) environment. Any of such aquatic environments, freshwater species, marine species, and/or species that thrive in varying and/or intermediate salinities or nutrient levels, can be used in certain embodiments. It is advantageous to use algae and planktivorous fishes from a local aquatic trophic system in the methods provided herein. The algae in an algal composition provided herein can be obtained initially from environmental samples of natural or man-made environments, and may contain a mixture of prokaryotic and eukaryotic organisms, wherein some of the species may be unidentified. Freshwater filtrates from rivers, lakes; seawater filtrates from coastal areas, oceans; water in hot springs or thermal vents; and lake, marine, or estuarine sediments, can be used to source the algae. The samples may also be collected from local or remote bodies of water, including surface as well as subterranean water. Endemic or indigenous algal species are generally preferred over introduced species.

In addition to using mass per unit volume (such as mg/l or g/l), chlorophyll a is a commonly used indicator of algal biomass. However, it is subjected to variability of cellular chlorophyll content (0.1 to 9.7% of fresh algal weight) depending on algal species. An estimated biomass value can be calibrated based on the chlorophyll content of the dominant species within a population. Published correlation of chlorophyll a concentration and biomass value can be used in certain embodiments. Generally, chlorophyll a concentration is to be measured within the euphotic zone of a body of water. The euphotic zone is the depth at which the light intensity of the photosynthetically active spectrum (400-700 nm) equals 1% of the subsurface light intensity.

One or more species of algae are present in the algal composition that is introduced into the water within the confines of the cages provided herein. In one embodiment, the algal composition is a monoculture, wherein only one species of algae is grown. However, in many open culturing systems, it may be difficult to avoid the presence of other algae species in the water. Accordingly, a monoculture may comprise about 0.1% to 2% cells of algae species other than the intended species, i.e., up to 98% to 99.9% of the algal cells in a monoculture are of one species. In certain embodiments, the algal composition comprise an isolated species of algae, such as an axenic culture.

In another embodiment, the algal composition is a mixed culture that comprises more than one species of algae, i.e., the algal culture is not a monoculture. The body of water that percolates the cages provided herein can contain a mix of different species of algae. Such an algal composition or a body of water can contain a naturally occurring assemblage of different species of algae or it can be prepared by mixing different algal cultures or axenic cultures. In certain embodiments, the algal composition or body of water can also comprise zooplankton, bacterioplankton, and/or other planktonic organisms. In certain embodiments, an algal composition comprising a combination of different batches of algal cultures is used in certain embodiments. The algal composition can be prepared by mixing a plurality of different algal cultures. The different taxonomic groups of algae can be present in defined proportions, which corresponds to the diversity of algal species in the local aquatic environment. The combination and proportion of different algae in an algal composition can be designed or adjusted to enhance the growth and/or accumulation of lipids of certain groups or species of fish. The combination and proportion of different algae present in the body of water in which the cages provided herein are situated can be one of the distinguishing characteristics of a system provided herein.

A microalgal composition provided herein can comprise microalgae of a selected size range, such as but not limited to, below 2000 μm, about 200 to 2000 μm, above 200 μm, below 200 μm, about 20 to 2000 μm, about 20 to 200 μm, above 20 μm, below 20 μm, about 2 to 20 μm, about 2 μm to 200 μm, about 2 to 2000 μm, below 2 μm, about 0.2 to 20 μm, about 0.2 to 2 μm or below 0.2 μm. An algal composition provided herein can be a concentrated algal culture or composition that comprises about 110%, 125%, 150%, 175%, 200% (or 2 times), 250%, 500% (or 5 times), 750%, 1000% (10 times) or 2000% (20 times) the amount of algae in the original culture or in a preceding algal composition.

A mixed algal composition provided herein comprises one or several dominant species of macroalgae and/or microalgae. Microalgal species can be identified by microscopy and enumerated by counting, microfluidics, or flow cytometry, which are techniques well known in the art. A dominant species is one that ranks high in the number of algal cells, e.g., the top one to five species with the highest number of cells relative to other species. Microalgae occur in unicellular, filamentous, or colonial forms. The number of algal cells can be estimated by counting the number of colonies or filaments. Alternatively, the dominant species can be determined by ranking the number of cells, colonies and/or filaments. This scheme of counting may be preferred in mixed cultures where different forms are present and the number of cells in a colony or filament is difficult to discern. In a mixed algal composition, the one or several dominant algae species may constitute greater than about 10%, about 20%, about 30%, about 40%, about 50%, about 60%, about 70%, about 80%, about 90%, about 95%, about 97%, about 98% of the algae present in the culture. In certain mixed algal composition, several dominant algae species may each independently constitute greater than about 10%, about 20%, about 30%, about 40%, about 50%, about 60%, about 70%, about 80% or about 90% of the algae present in the culture. Many other minor species of algae may also be present in such composition but they may constitute in aggregate less than about 50%, about 40%, about 30%, about 20%, about 10%, or about 5% of the algae present. In various embodiments, one, two, three, four, or five dominant species of algae are present in an algal composition. Accordingly, a mixed algal culture or an algal composition can be described and distinguished from other cultures or compositions by the dominant species of algae present. An algal composition can be further described by the percentages of cells that are of dominant species relative to minor species, or the percentages of each of the dominant species. The identification of dominant species can also be limited to species within a certain size class, e.g., below 2000 μm, about 200 to 2000 μm, above 200 μm, below 200 μm, about 20 to 2000 μm, about 20 to 200 μm, above 20 μm, below 20 μm, about 2 to 20 μm, about 2 to 200 μm, about 2 to 2000 μm, below 2 μm, about 0.2 to 20 μm, about 0.2 to 2 μm or below 0.2 μm. It is to be understood that mixed algal compositions or a body of water having the same genus or species of algae may be different by virtue of the relative abundance of the various genus and/or species that are present.

It is contemplated that many different algal compositions or bodies of water which comprise plankton, can be used in the methods provided herein. Microalgae are preferably used in many embodiments provided herein; while macroalgae are less preferred in certain embodiments. In specific embodiments, algae of a particular taxonomic group, e.g., a particular genera or species, may be less preferred in a culture. Such algae, including one or more that are listed below, may be specifically excluded as a dominant species in a culture or composition. However, it should also be understood that in certain embodiments, such algae may be present as a contaminant, a non-dominant group or a minor species, especially in an open system. Such algae may be present in negligent numbers, or substantially diluted given the volume of the culture or composition. The presence of such algal genus or species in a culture, composition or a body of water is distinguishable from cultures, composition or bodies of water where such algal genus or species are dominant, or constitute the bulk of the algae. The composition of an algal culture or a body of water in an open culturing system is expected to change according to the four seasons, for example, the dominant species in one season may not be dominant in another season. An algal composition or a body of water at a particular geographic location or a range of latitudes can be more specifically described by season, i.e., spring composition, summer composition, fall composition, and winter composition; or by any one or more calendar months, such as but not limited to, from about December to about February, or from about May to about September.

In certain embodiments, an algal composition comprising a combination of different groups of algae is used in certain embodiments. The algal composition can be prepared by mixing a plurality of different algal cultures. The different groups of algae can be present in defined proportions. The combination and proportion of different algae in the algal composition can be designed to enhance the growth of certain groups or species of fish.

In various embodiments, one or more species of algae belonging to the following phyla can be used in the methods provided herein: Cyanobacteria, Cyanophyta, Prochlorophyta, Rhodophyta, Glaucophyta, Chlorophyta, Dinophyta, Cryptophyta, Chrysophyta, Prymnesiophyta (Haptophyta), Bacillariophyta, Xanthophyta, Eustigmatophyta, Rhaphidophyta, and Phaeophyta. In certain embodiments, algae in multicellular or filamentous forms, such as seaweeds or macroalgae, many of which belong to the phyla Phaeophyta or Rhodophyta, are less preferred. In certain embodiments, algae that are microscopic, are preferred. Many such microalgae occurs in unicellular or colonial form.

The algal composition can comprises cyanobacteria (also known as blue-green algae) from one or more of the following taxonomic groups: Chroococcales, Nostocales, Oscillatoriales, Pseudanabaenales, Synechococcales, and Synechococcophycideae. Non-limiting examples include Gleocapsa, Pseudoanabaena, Oscillatoria, Microcystis, Synechococcus and Arthrospira species.

The algal composition can comprises algae from one or more of the following taxonomic classes: Euglenophyceae, Dinophyceae, and Ebriophyceae. Non-limiting examples include Euglena species and the freshwater or marine dinoflagellates.

The algal composition can comprises green algae from one or more of the following taxonomic classes: Micromonadophyceae, Charophyceae, Ulvophyceae and Chlorophyceae. Non-limiting examples include species of Borodinella, Chlorella (e.g., C. ellipsoidea), Chlamydomonas, Dunaliella (e.g., D. salina, D. bardawil), Franceia, Haematococcus, Oocystis (e.g., O. parva, O. pustilla), Scenedesmus, Stichococcus, Ankistrodesmus (e.g., A. falcatus), Chlorococcum, Monoraphidium, Nannochloris and Botryococcus (e.g., B. braunii). In certain embodiments, Chlamydomonas reinhardtii are less preferred.

The algal composition can comprises golden-brown algae from one or more of the following taxonomic classes: Chrysophyceae and Synurophyceae. Non-limiting examples include Boekelovia species (e.g. B. hooglandii) and Ochromonas species.

The algal composition can comprises freshwater, brackish, or marine diatoms from one or more of the following taxonomic classes: Bacillariophyceae, Coscinodiscophyceae, and Fragilariophyceae. Preferably, the diatoms are photoautotrophic or auxotrophic. Non-limiting examples include Achnanthes (e.g., A. orientalis), Amphora (e.g., A. coffeiformis strains, A. delicatissima), Amphiprora (e.g., A. hyaline), Amphipleura, Chaetoceros (e.g., C. muelleri, C. gracilis), Caloneis, Camphylodiscus, Cyclotella (e.g., C. cryptica, C. meneghiniana), Cricosphaera, Cymbella, Diploneis, Entomoneis, Fragilaria, Hantschia, Gyrosigma, Melosira, Navicula (e.g., N. acceptata, N. biskanterae, N. pseudotenelloides, N. saprophila), Nitzschia (e.g., N. dissipata, N. communis, N. inconspicua, N. pusilla strains, N. microcephala, N. intermedia, N. hantzschiana, N. alexandrina, N. quadrangula), Phaeodactylum (e.g., P. tricornutum), Pleurosigma, Pleurochrysis (e.g., P. carterae, P. dentata), Selenastrum, Surirella and Thalassiosira (e.g., T. weissflogii).

In various embodiments, the algal composition comprises planktons that are characteristically small with a diameter in the range of 1 to 10 μm, or 2 to 4 μm. Many of such algae are members of Eustigmatophyta, such as but not limited to Nannochloropsis species (e.g. N salina).

In various embodiments, the algal composition comprises one or more algae from the following groups: Coelastrum, Chlorosarcina, Micractinium, Porphyridium, Nostoc, Closterium, Elakatothrix, Cyanosarcina, Trachelamonas, Kirchneriella, Carteria, Crytomonas, Chlamydamonas, Planktothrix, Anabaena, Hymenomonas, Isochrysis, Pavlova, Monodus, Monallanthus, Platymonas, Pyramimonas, Stephanodiscus, Chroococcus, Staurastrum, Netrium, and Tetraselmis.

In certain embodiments, any of the above-mentioned genus and species of algae may independently be less preferred as a dominant species in, or excluded from, an algal composition provided herein.

All references cited herein are incorporated herein by reference in their entirety and for all purposes to the same extent as if each individual publication or patent or patent application was specifically and individually indicated to be incorporated by reference in its entirety for all purposes.

Many modifications and variations of the embodiments provided herein can be made without departing from its spirit and scope, as will be apparent to those skilled in the art. The specific embodiments described herein are offered by way of example only, and are to be limited only by the terms of the appended claims along with the full scope of equivalents to which such claims are entitled.

1. An aquaculture system comprising:
(i) a first cage and a second cage that are independently buoyant and closely disposed to each other;
(ii) an array of spaced rotary panels disposed between said first cage and second cage, wherein said panels, each being rotatable about a vertical or horizontal axis, increase or decrease gaps between neighboring panels, thereby regulating the flow of matters from the first cage to the second cage; and
(iii) a means for producing a directional water current in the first cage.

2. The system of claim 1, wherein the first cage is disposed inside the second cage, such that the exterior surface of the first cage and the interior surface of the second cage define a space for culturing fishes and/or shellfishes.

3. The system of claim 2, wherein said panels in said array are held vertically, equidistantly spaced along the perimeter of the inner cage, and rotatable in unison around their vertical axes.

4. The system of claim 2, wherein said panels in said array are held horizontally, equidistantly spaced from the top to the bottom of the array, and rotatable in unison around their horizontal axes.

5. The system of claim 2, wherein the first cage and the second cage are independently, prismatic, cylindrical, or funnel-shaped, and are aligned coaxially.

6. The system of claim 1, wherein the second cage comprises a second array of spaced rotary panels, said panels each being rotatable about a vertical or horizontal axis to increase or decrease gaps between neighboring panels which regulate the flow of water from the second cage to ambient water.

7. The system of claim 1, wherein a third cage or a receptacle is closely disposed to the bottom of the first cage and/or the bottom of the second cage, such that solid wastes in the first cage and/or second cage are directed into the third cage or the receptacle.

8. The system of claim 1, wherein the means for producing directional water current is a water pump, an air pump, a venturi aerator, an agitator, a propeller, or a columnar air/water jet diffuser.

9. An aquaculture system comprising:
(i) a buoyant cylindrical inner cage which contains piscivorous fishes or feed-fed fishes, said inner cage being disposed coaxially inside a buoyant outer cage which contains algae, and planktivorous fishes and/or shellfishes in a space defined by exterior surfaces of the inner cage and interior surfaces of the outer cage, said outer cage having a diameter that is about two to five times the diameter of said inner cage;
(ii) an array of equidistantly spaced rotary panels, said array being disposed between said inner and outer cage, and wherein said panels, each being rotatable about a vertical or horizontal axis, increase or decrease gaps between neighboring panels, thereby regulating the flow of matters from said inner cage to said outer cage; and
(iii) one or more columnar air/water jet diffuser disposed within the inner cage and which produces a directional water current in the inner cage, thereby driving the lateral flow of water from the inner cage to the outer cage.

10. A method of aquaculture comprising:
(i) providing an aquaculture system comprising a first cage and a second cage that are independently buoyant and closely disposed to each other;
(ii) providing an array of spaced rotary panels disposed between said first cage and second cage, wherein said panels, each being rotatable about a vertical or horizontal axis, increase or decrease gaps between neighboring panels, thereby regulating the flow of matters from the first cage to the second cage;
(iii) providing piscivorous fishes and/or feed-fed fishes in the inner cage, and algae and planktivorous fishes and/or shellfishes in a space defined by exterior surfaces of the inner cage and interior surfaces of the outer cage;
(iv) rotating the panels periodically to an angular position that reduces or prevents passage of solid wastes from the inner cage to the outer cage through gaps between the panels; and
(v) rotating the panels periodically to an angular position that increases or allows flow of water from the inner cage to the outer cage through the gaps between the panels.

11. The method of claim 10, further comprising:
feeding the piscivorous fishes and/or feed-fed fishes in said inner cage with the planktivorous fishes in said outer cage, or fish feed that comprises the planktivorous fishes in said outer cage.

12. The method of claim 10, wherein said aquaculture system is disposed in a body of eutrophic water, and the algae grown in the outer cage using nutrients in the body of eutrophic water.

13. The method of claim 10, further comprising:
feeding the planktivorous fishes and/or shellfishes in said inner cage with an algal composition.

14. The method of claim 10, further comprising:
harvesting the piscivorous fishes and/or feed-fed fishes.

15. The method of claim 10, further comprising:
harvesting the piscivorous fishes and/or feed-fed fishes, and the planktivorous fishes and/or shellfishes.

16. The method of claim 10, further comprising:
processing the planktivorous fishes and/or viscera of piscivorous fishes and/or detritivorous fishes into fishmeal, fish oil, fish lipids, and/or fish protein concentrate.

17. The method of claim 16, further comprising:
processing said fish oil or lipids to form a biofuel feedstock.

18. The method of claim 16, further comprising:
processing said fish oil or lipids to form biofuel, biodiesel, fatty acid methyl ester, diesel, kerosene, a jet-fuel, gasoline, JP-1, JP-4, JP-5, JP-6, JP-7, JP-8, or JPTS.

19. The method of claim 16, further comprising:
processing said fish oil or lipids to form EPA- and/or DHA-containing lipids or substantially purified EPA and/or DHA.

20. The method of claim 19, wherein said processing step comprises a processing technique selected from chromatography, fractional or molecular distillation, enzymatic splitting, low-temperature crystallization, supercritical fluid extraction, or urea complexation.

21. The method of claim 16, further comprising:
processing said fish oil or lipids to form EPA- and/or DHA-containing products for human consumption or animal feeds.

22. The method of claim 21, wherein said processing step comprises a processing technique selected from chromatography, fractional or molecular distillation, enzymatic splitting, low-temperature crystallization, supercritical fluid extraction, or urea complexation.

23. The method of claim 10, wherein said aquaculture system further comprises a bottom cage disposed closely to said inner cage and/or said outer cage, said bottom cage comprising detritivorous fishes that consume solid wastes produced in said inner cage and/or said outer cage.

24. The method of claim 10, wherein said aquaculture system further comprises a receptacle connected to a sludge pump for conveying solid wastes.

25. The method of claim 10, wherein the fishes in the inner cage are red snapper (Lutjanus campechanus), snook (Centropomus undecimalis), white sturgeon (Acipenser transmontanus), pompano (Trachinotus carolinus), red grouper (Epinephelus morio), red drum (Sciaenops ocellatus), barramundi (Lates calcarifer), or a combination of two or more the fish species.

26. The method of claim 10, wherein the fishes in the outer cage are Atlantic Herring (Clupea harengus harengus), Gulf Menhaden (Brevoortia patronus), or Northern anchovy (Engraulis mordax) or a combination of two or more the fish species.

27. The method of claim 10, wherein the shellfishes are oysters, mussels, scallops, clams, or a combination of two or more of the shellfish species.

28. The method of claim 10, wherein the detritivorous fishes are marine catfish, mullets, black drum or a combination of two or more of the fish species.

29. The system of claim 2, wherein the means for producing directional water current is a water pump, an air pump, a venturi aerator, an agitator, a propeller, or a columnar air/water jet diffuser.

30. The method of claim 18, wherein said processing step comprises transesterification, acid-catalyzed transesterification, base-catalyzed transesterification, enzyme-catalyzed transesterification, supercritical methanol transesterification, hydrogenation, hydrocracking, and/or isomerization.
